(19) United States
(12) Patent Application Publication
(10) Pub. No.: US 2018/0078661 A1
(43) Pub. Date: Mar. 22, 2018

(54) IMAGEABLE BIORESORBABLE EMBOLIZATION MICROSPHERES

(71) Applicant: Regents of the University of Minnesota, Minneapolis, MN (US)

(72) Inventors: Lihui Weng, Woodbury, MN (US); Jafar Golzarian, Plymouth, MN (US)

(21) Appl. No.: 15/709,124

(22) Filed: Sep. 19, 2017

Related U.S. Application Data
(60) Provisional application No. 62/397,237, filed on Sep. 20, 2016.

(51) Int. Cl. A61K 49/18; A61K 31/704

(52) U.S. Cl. CPC A61K 49/1863; A61K 31/704

(57) ABSTRACT

The present disclosure is generally directed to an embolic material which, in some examples, may be in the form of a microsphere or a plurality of microspheres. The embolic material may include carboxymethyl chitosan (CCN) crosslinked with partially oxidized carboxymethyl cellulose (OCMC) and an imaging agent such as at least one of an ethiodized oil, a radiopaque metal, or super paramagnetic iron oxide nanoparticles integrally contained within the microsphere.

This application claims the benefit of U.S. Provisional Patent Application No. 62/397,237, filed Sep. 20, 2016, the entire content of which being incorporated herein by reference.

### TECHNICAL FIELD

The disclosure relates to embolic materials.

### BACKGROUND

Transcatheter arterial embolization (TAE) has been widely accepted for its efficacy in treating various diseases including tumors, vascular lesions, and hemorrhages. For a safe and effective treatment, the selection of an appropriate embolic material is important.

### SUMMARY

The present disclosure is generally directed to an embolic material which, in some examples, may be in the form of a microsphere or a plurality of microspheres. The microspheres may include carboxymethyl chitosan (CCN) crosslinked with at least partially oxidized carboxymethyl cellulose (OCMC). The microspheres may also include an imaging agent such as at least one of an ethiodized oil, a radiopaque metal, or superparamagnetic iron oxide nanoparticles (SPIONs), integrally contained within the microsphere. The imaging agent may improve the visibility of the microspheres during certain imaging techniques including, for example, magnetic resonance imaging (MM), X-ray imaging, X-ray computed tomography (CT) imaging, fluoroscopy, or the like.

In some examples, the disclosure describes an embolic material that includes a microsphere defining a diameter between about 50 micrometers and about 2200 micrometers, where the microsphere includes CCN crosslinked with OCMC, and an imaging agent integrally contained within the microsphere.

In some examples, the disclosure describes an embolization suspension that includes a solvent and a plurality of microspheres suspended in the solvent. At least one microsphere of the plurality of microspheres defines a diameter between about 50 micrometers and about 2200 micrometers and includes CCN crosslinked with OCMC and an imaging agent integrally contained within the microsphere.

In some examples, the disclosure describes a method of forming an embolic microsphere that includes at least partially oxidizing CMC to form OCMC, forming an emulsion of OCMC, CCN, an aqueous solvent, an imaging agent, and a non-aqueous solvent, and crosslinking the CCN with the OCMC to form the embolic microsphere, where the imaging agent is integrally contained within the embolic microsphere.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a flow diagram of an example technique for producing embolic microspheres comprising CCN crosslinked with OCMC.

FIGS. 2A and 2B are a photograph and a light microscopy image, respectively, of microspheres in accordance with one aspect of the disclosure.

FIG. 3 is an example of a scanning electron microscopy image of an exterior of a microsphere formed in accordance with aspects of this disclosure.

FIGS. 4A and 4B are examples of SEM images of the cross-section of a hydrogel prepared with CCN crosslinked with OCMC.

FIG. 5A-5G are images of microspheres formulated in accordance with Table 1.

FIGS. 6A-6C are fluoroscopic images taken of microspheres formed in accordance with the formulas of Table 1.

FIGS. 7A-7C are light microscopy images illustrating an example of the compressibility of a microsphere comprising CCN crosslinked with OCMC (dyed with Evan's blue) as the microsphere passes through a polyethylene tube.

FIGS. 8A-8C are light microscopy images that illustrate another example of the compressibility of a microsphere comprising CCN crosslinked with OCMC (dyed with Evan's blue) as the microsphere passes through a polyethylene tube.

FIGS. 9A and 9B illustrate an example of the resiliency of a microsphere comprising CCN crosslinked with OCMC (dyed with Evan's blue).

FIG. 10 is a light microscopy image of an example of microspheres comprising CCN crosslinked with OCMC and having diameters between about 500 μm and about 700 μm after being injected through a catheter with an internal diameter of about 667 μm (2 French).

FIGS. 11A and 11B are light microscopy images of an example of microspheres comprising CCN crosslinked with OCMC and having diameters between about 800 μm and about 1000 μm after being injected through a catheter with an internal diameter of about 1 mm (3 French).

FIGS. 12A and 12B are light microscopy images of an example of microspheres comprising CCN crosslinked with OCMC and loaded with doxorubicin while passing through a catheter and after passing through the catheter.

FIGS. 13A and 13B are light microscopy images of an example of microspheres comprising CCN crosslinked with OCMC and loaded with doxorubicin while passing through a polyethylene tube.

FIG. 14 is a light microscopy image of an example of a plurality of microspheres comprising CCN crosslinked with OCMC after being stored for two months in water.

FIGS. 15A-15E are light microscopy images that illustrate an example of a plurality of microspheres comprising CCN crosslinked with OCMC degrading in the presence of lysozyme.

FIG. 16 is a bar graph that illustrates an example of the arterial distribution of the microspheres after embolization of three pairs of rabbit kidneys with microspheres having diameters between about 100 μm and about 300 μm.

FIG. 17 is bar diagram that illustrates an example comparison between Embospheres® and microspheres formed of CCN crosslinked with OCMC of the mean diameter of the vessel occluded during an embolization procedure.

FIG. 18 is a bar diagram that illustrates an example determination of the mean diameter of the microspheres formed of CCN crosslinked with OCMC that were used in the embolization procedure that generated the results shown in FIG. 18.

FIGS. 19 and 20 are example histology sections of kidney tissue showing arcuate artery in a kidney of a rabbit occluded with an embolic microsphere according to aspects of the disclosure.

FIG. 21A is a light microscopy image illustrating the appearance of MRI visible microspheres having diameters between about 300 μm and about 500 μm.

FIG. 21B is a light microscopy image illustrating the appearance of MRI visible microspheres having diameters between about 700 μm and about 900 μm.

FIGS. 22A-22C are fluorescent confocal microscopy images of an Mill visible microsphere at a top focal plane, a middle focal plane, and an equatorial focal plane.

FIG. 23 is an image illustrating an arrangement of samples within an MRI modality.

FIGS. 24A-24C are MM images of MRI visible microsphere samples collected using different Mill imaging techniques.

FIGS. 25A and 25B are plots of relaxivity (R1, measured in s⁻¹) versus SPIO concentration for MM visible microspheres.

FIG. 26 is a plot of percent doxorubicin released versus time for doxorubicin-loaded Mill visible microspheres.

FIG. 27 is a light microscopy image showing degraded doxorubicin loaded Mill visible microspheres after 13 days in 0.01M PBS at 37° C.

### DETAILED DESCRIPTION

The present disclosure is generally directed to an embolic material that, in some examples, may be in the form of a microsphere or a plurality of microspheres. Microspheres may be useful to provide temporary embolization within a patient, particularly when the microspheres are spherical, biocompatible, bioresorbable, and compressible. However, these properties are not easily achieved with single embolic material or microsphere. For example, microspheres may be formed by crosslinking various polymers used to form the microspheres. Such crosslinking may be accomplished by using a small molecule crosslinking agent, such as glutaraldehyde. While use of the small molecule crosslinking agent facilitates the desired crosslinking reaction, if the crosslinked polymer is biodegradable and degrades in a body of a patient, some the small-molecule crosslinking agents may be released leading to adverse effects on cells or tissue in the body of the patient.

In accordance with aspects of this disclosure, the embolic material used to form the microspheres may include CCN crosslinked with OCMC.

In some examples, the CCN and OCMC may be crosslinked without the use of a small molecule crosslinking agent to form embolic microspheres that are substantially free of a small molecule crosslinking agent. In fact, in some examples, the crosslinking reaction between OCMC and CCN may be carried out without a small molecule crosslinking agent at relatively low temperatures (e.g., about 50° C.) in a water and oil emulsion. CCN is substantially non-toxic and biodegradable within the body of a patient. For example, the CCN may break down within the body of a patient to glucosamine, which can be substantially completely absorbed by a patient's body without adverse effects. Similarly, OCMC is substantially non-toxic and biodegradable within the body of a patient. Thus, a crosslinked polymer formed by CCN and OCMC is expected to be substantially non-toxic (i.e., biocompatible) and biodegradable (or bioresorbable). Additionally, because the crosslinked CCN and OCMC microspheres are formed from two polymers, the mechanical properties, such as compressibility, of the crosslinked molecule are sufficient for injection of the microspheres through a syringe or catheter.

While the resultant microspheres demonstrate good properties as an embolic material, the CCN and OCMC composition of the microspheres renders them substantially radiolucent or non-imageable within the body of a patient, which can make it difficult to visualize the microspheres after introduction into a body of a patient to determine proper placement and distribution within the body. In some examples, the microspheres may be combined (e.g., soaked) in a surgical contrast in order enhance the imaging properties of the microsphere solution. With the inclusion of such contrasting agents, a physician may be able to monitor the injection of the microspheres using, for example, fluoroscopy to determine the spheres placement or distribution within the patient. However, the surgical contrast may not allow for a precise imaging location (e.g., edge identification) of the microspheres due to the transfer of the contrast into the carrier agent. Such lack of precise imaging location may lead to improper identification of the location of the embolic microspheres, which can cause non-target embolization. Furthermore, over time, such as a matter of about 1 day to about 2 days, the contrast solution may be substantially washed out of the embolic microspheres within the body of a patient, thereby reducing or eliminating any imaging benefits obtained by using the contrast solution. Over time, the diminished imageability may result in difficulty tracking these microspheres sufficiently at follow-up patient consultations as the microspheres are no longer visible under imaging procedures such as fluoroscopy, MRIs, CTs, x-rays, and the like.

In accordance with aspects of this disclosure, the microspheres described herein may include an imaging agent within the microsphere that may be used to enhance the viewability (e.g., imaging contrast) of the microspheres within the body of a patient during different types of medical imaging procedures including, for example, MRI scans, X-ray imaging, CT scans, fluoroscopy, and the like. In some examples, the imaging agent may be integrally contained within the microspheres such that the imaging agent is not easily removed or washed from the microspheres, apart from degradation of the microsphere. This may result in the imaging agent remaining within the microsphere throughout the life of the microspheres, e.g., the microspheres may remain imageable within the body of a patient even after partial degradation. For example, the imaging agent may be confined within the resultant CCN/OCMC polymer network allowing the microsphere to remain visible within the body of a patient for an extended period of time. In some examples, the imaging agent may permit the microspheres to remain imageable within the body of a patient until the microspheres have fully degraded. The degradation time may be influenced by the composition of the beads, e.g., including the extent of crosslinking.

In some examples, imaging agent may remain fixed within the microspheres so that the microspheres may be observed under MM, X-ray imaging, CT, fluoroscopy, or the like until the microspheres have substantially disintegrated within the patient's body (e.g., fully degraded or degraded so that only a minuscule amount remains). In some examples, the enhanced imageability of the microspheres may significantly reduce non-target embolization during administration of the microspheres and provide improved tracking of the microspheres while they remain in the patient's body. The imaging agent may include, for example, an ethiodized oil, a radiopaque metal, or superparamagnetic iron oxide nanoparticles (SPIONs).

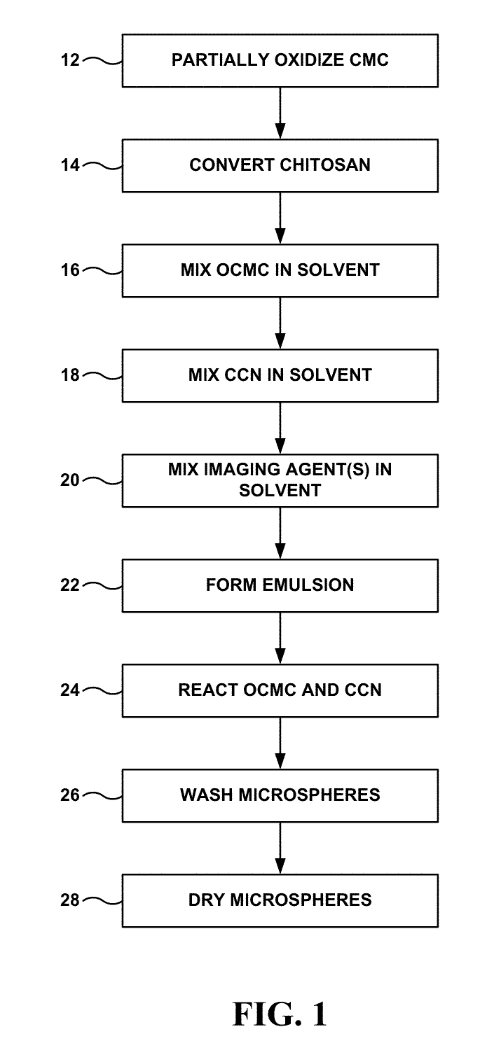

In some examples, the microspheres comprising CCN and OCMC may be formed according to the technique illustrated in FIG. 1. Initially, CMC is at least partially oxidized to form OCMC (12). One reaction that at least partially oxidizes CMC is illustrated in Reaction 1:

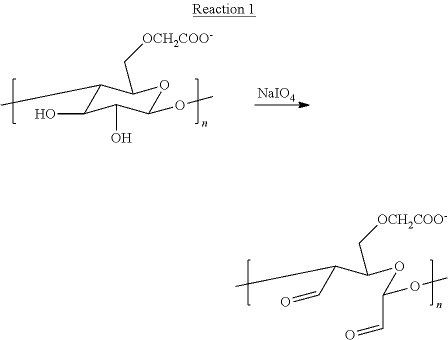

In Reaction 1, a single CMC monomer (repeating unit), which is part of a chain comprising n repeating units, is reacted with NaIO₄ (sodium periodate) at about 25° C. to oxidize the C—C bond between carbon atoms bonded to hydroxyl groups to form carbonyl (more particularly aldehyde) groups. Reaction 1 shows only a single repeating unit of the CMC polymer. In some examples, not all repeating units within the CMC polymer may be oxidized. For example, some repeating units may not be oxidized at all, and may still include two hydroxyl groups after Reaction 1 is performed. Other monomers may be oxidized, and may include two carbonyl groups, as illustrated in Reaction 1. The CMC may include a weight average molecular weight of between about 50,000 daltons (Da; equivalent to grams per mole (g/mol)) and about 800,000 Da. In some examples, a weight average molecular weight of the CMC may be about 700,000 g/mol.

The degree of oxidation of the CMC may be affected by, for example, the molar ratio of NaIO₄ to CMC repeating units. In some examples, the molar ratio of NaIO₄ molecules to CMC repeating units may be between about 0.1:1 and about 0.5:1 (NaIO₄:CMC). Particular examples of molar ratios of NaIO₄ molecules to CMC repeating units include about 0.1:1, about 0.25:1, and about 0.5:1. An increased molar ratio of NaIO₄ molecules to CMC repeating units may result in greater oxidation of the CMC, which in turn may lead to greater crosslinking density when CMC is reacted with CCN to form the embolic microspheres. Conversely, a decreased molar ratio of NaIO₄ molecules to CMC repeating units may result in lesser oxidation of the CMC, which in turn may lead to lower crosslinking density when CMC is reacted with CCN to form the embolic microspheres. In some examples, the crosslinking density may be approximately proportional to the degree of oxidation of the CMC. In some examples, a greater crosslinking density may lead to greater mechanical strength (e.g., fracture strain). In some examples, rather than at least partially oxidizing CMC to form OCMC (12), the technique of FIG. 1 may include obtaining OCMC, for example, from a supplier.

The technique of FIG. 1 then may include preparing CCN by reacting chitosan to attach —CH₂COO⁻ groups in place of one of the hydrogen atoms in an amine group or a hydroxyl group, as illustrated in Reaction 2 (14).

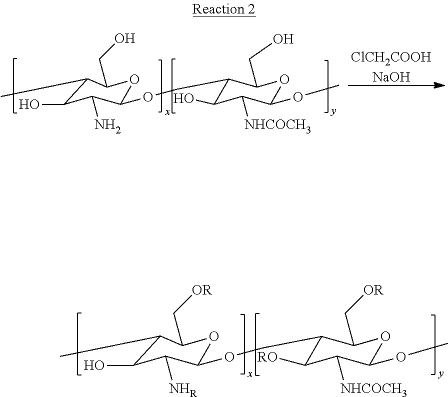

In the product of Reaction 2, each R is independently either H or —CH₂COO⁻. Similar to oxidation of CMC shown in Reaction 1, the extent of the addition of the —CH₂COO⁻ may affect the crosslink density when the CCN is reacted with the OCMC to form the embolic microspheres. In some examples, the ratio of x:y may be about 3:1 (i.e., monomers of “x” form about 75% of the chitosan and monomers of “y” form about 25% of the chitosan). In some examples, the chitosan starting material may have a molecular weight between about 190,000 g/mol and about 375,000 g/mol. In some examples, Reaction 2 may be performed by stirring the reaction mixture at 500 rpm for about 24 hours at about 25° C., followed by stirring the reaction mixture at 500 rpm for about 4 hours at about 50° C.

Once the OCMC and the CCN have been prepared, each may be mixed in a respective amount of an aqueous solvent, such as water (16), (18). For example, 0.1 milligram (mg) of OCMC may be mixed in 5 milliliter (mL) of water to form a first 2% weight/volume (w/v) solution. Similarly, 0.1 mg of CCN may be mixed in 5 mL of water to form a second 2% w/v solution. While the solvent is described primarily as water, aqueous solvents other than water may also be used. For example, saline or phosphate-buffered saline (PBS) may be utilized as alternative solvents. The aqueous solvent used for the OCMC solution may be the same as or different than the solvent used for the CCN solution. The solutions may have concentrations of OCMC or CCN between about 0.5% w/v and about 3% w/v. The concentration of the OCMC solution may be the same as or different from the concentration of the CCN solution. Additionally, or alternatively, solutions having other concentrations of OCMC or CCN may be utilized. In some examples, rather than preparing CCN by reacting chitosan to attach —CH₂COO⁻ groups in place of one of the hydrogen atoms in an amine group or a hydroxyl group (14), the technique of FIG. 1 may include obtaining CCN, for example, from a supplier.

The technique of FIG. 1 also includes adding an imaging agent to one or more of the respective aqueous solvents containing the OCMC and the CCN (20). The imaging agent may be added to the aqueous solvent either before, after, or concurrently with the addition of the OCMC or CCN to the respective solvent. In some examples, the imaging agent may be mixed with CCN in the aqueous solvent to form part of the CCN solution.

Any suitable non-toxic imaging agent may be added to the aqueous solvent. Suitable imaging agents may include, for example, ethiodized oils such as Lipiodol® (available from Guerbet LLC, 120 W. 7th Street, Suite 109, Bloomington, Ind. 47404 and manufactured for Guerbet LLC by Jubilant HollisterStier General Partnership, 16751 Trans-Canada Highway, Kirkland, Quebec, Canada H9H 4J4); radiopaque metals such as tantalum, tungsten, barium, bismuth, platinum, gold, or the like; a paramagnetic or superparamagnetic material such as superparamagnetic iron oxide nanoparticles (SPIONs), or the like.

In some examples, the imaging agent may include at least one ethiodized oil (e.g. Lipiodol®) or may consist essentially of one or more ethiodized oils (e.g., consist of one or more ethiodized oils and any other contaminates/compounds that do not materially affect the basic characteristics of the ethiodized oils and imaging agent). The ethiodized oil may provide sufficient contrast characteristics to the resultant microspheres without significantly impacting the density (and thus weight) of the resultant microspheres. As a result, microspheres formed using at least one ethiodized oil remain comparatively light, e.g., less dense, and are less prone to clumping or settling prior to administration. In some examples, the ethiodized oil may be added to one or more of the CCN/CMC aqueous mixtures (e.g., CCN in aqueous solvent) at a volumetric ratio of about 1 part ethiodized oil(s) to 1 part aqueous solvent of one of the CCN/CMC aqueous mixtures. The at least one ethiodized oil may improve imaging of the microspheres during certain imaging techniques including, for example, CT imaging, fluoroscopy, or the like.

In some examples, the imaging agent may include at least one ethiodized oil in combination with one or more radiopaque metals (e.g., tantalum, tungsten, barium, bismuth, platinum, gold, or the like). In some examples, the combination of ethiodized oil with one or more radiopaque metals may provide an improved contrast resolution compared to an imaging agent consisting of the ethiodized oil or radiopaque metal independently. Additionally, or alternatively, the combination of ethiodized oil and radiopaque metal may allow the density of the imaging agent to remain relatively low compared to an imaging agent consisting of only radiopaque metals.

In some such examples, the imaging agent may be prepared prior to adding the imaging agent to the aqueous solvent by mixing a powder form of the radiopaque metal(s) (e.g., tantalum, tungsten, barium, bismuth, platinum, gold, or the like) into the ethiodized oil so that the radiopaque metal(s) is sufficiently wetted and dispersed within the ethiodized oil. In some examples, the ratio the radiopaque metal(s) (e.g., tantalum, tungsten, barium, bismuth, platinum, gold, or the like) to the ethiodized oil may be about three parts by weight radiopaque metal(s) and about one part by weight ethiodized oil.

In other examples, the imaging agent may include or consist essentially of one or more paramagnetic materials (e.g., consist of one or more paramagnetic materials and any other contaminates/compounds that do not materially affect the basic and characteristics of the paramagnetic materials and imaging agent), such as SPIONs, gadolinium ions, or the like. In some examples, the paramagnetic materials may be in the form of nanoparticles that define an average particle diameter of about 10 nanometers (nm) to about 35 nm. Imaging agents that include paramagnetic materials may be useful to improve imaging of the microspheres during certain imaging techniques including, for example, MRI imaging. Being visible under MM may be useful for the detection of microspheres within the patient and assessment of the extent of embolization, thereby providing a valuable tool to predict the clinical outcome and safety of the procedure.

In some examples, the selection of imaging agent may depend on the desired size of the produced microspheres, intended use, or intended imaging technique. For example, the techniques described herein may be used to form imageable microspheres that have an average particle size between about 50 micrometers (μm) and about 2200 μm. In some examples where the desired size of the microspheres is greater than, for example, about 300 ethiodized oil or SPIONs may be used to provide sufficient imageability to the resultant microspheres. In other examples where the desired size of the microspheres is less than, for example, about 300 μm, SPIONs or a combination of ethiodized oil and radiopaque metal(s) may be used to provide sufficient imageability to the resultant microsphere. The combination of ethiodized oil(s) and radiopaque metal(s) may provide improved contrast resolution for the relatively small microspheres compared to either component alone, without significantly increasing the density of the resultant microspheres. Additionally, or alternatively, where MRI or X-ray imaging is the preferred imaging technique to be used, SPIONs may be used as the imaging agent.

Any appropriate amount of imaging agent may be added to one or both of the aqueous mixtures (e.g., CCN solution or OCMC solution) to provide sufficient contrast resolution to the resultant microspheres. In some examples, the resultant mixture may include about 5 percent to about 70 percent weight by volume (% w/v) of the weight of the imaging agent to the total volume of the aqueous solvents (e.g., the amount of solvents used to prepare both the OCMC and CCN materials). In some examples in which the imaging agent includes SPIONs, the resultant mixture may include between about 2 percent and about 10 percent volume by volume (% v/v) of the SPIONs.

Once the respective mixtures containing OCMC, CCN, and the desired imaging agent(s) have been prepared, the first and second mixtures may be added to another non-aqueous solvent to form an emulsion (22). In some examples in which water is utilized as the aqueous solvent for the OCMC, CCN, and imaging agent, the non-aqueous solvent may be an oil, such as, for example, mineral oil. In some examples, the non-aqueous solvent may include a surfactant mixed therein. One example of a suitable surfactant includes sorbitan monooleate, available under the tradename S6760 or Span® 80 from Sigma-Aldrich, St. Louis, Mo. In one example, 0.5 mL of sorbitan monooleate may be mixed in 50 mL of mineral oil, which is then mixed with the 5 mL 2% w/v solution of OCMC and the 5 mL 2% w/v solution of CCN.

The emulsion may be left for about 12 hours to about 16 hours (e.g., at least overnight) to allow the OCMC and CCN to react (24) in a modified emulsion-crosslinking reaction. In particular, an amino group on the CCN may react with an aldehyde group on the OCMC to form a Schiff base (i.e., an N═C double bond) and crosslink the OCMC and the CCN. One such crosslinking reaction is shown below in Reaction 3.

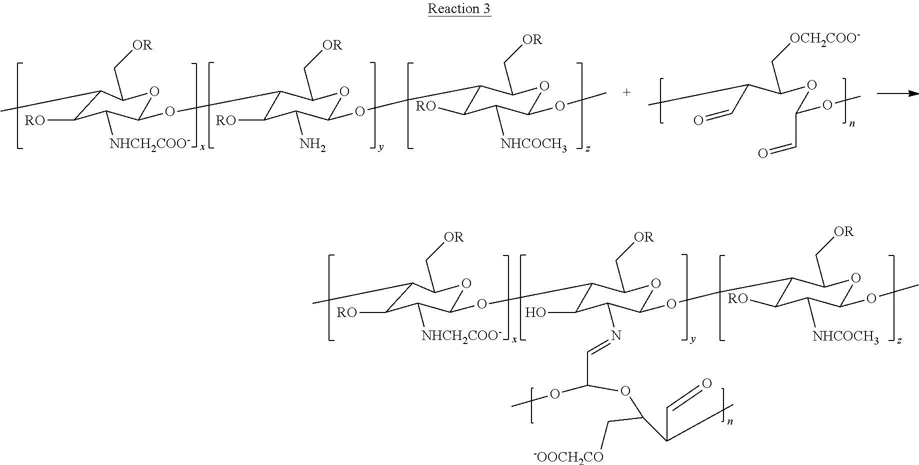

As discussed above, the crosslinking reaction of the OCMC and CCN may proceed without use of a small-molecule crosslinking agent, such as glutaraldehyde. This may be advantageous, because in some examples, the patient may be adverse to a small-molecule crosslinking agent used in the formation of the embolic microspheres. In this way, the microspheres formed from CCN crosslinked with OCMC may be substantially free of any small-molecule crosslinking agent.

As the crosslinking reaction process progresses to form the resultant microspheres, the imaging agent in the aqueous solvent solution(s) may become integrally contained within the forming microspheres. For example, the imaging agent may be confined within the resultant CCN/CMC polymer network such that the imaging agent becomes fixed within the polymer structure. In some examples, because the imaging agent is present during the formation of the respective microspheres and is integrally contained within the forming microspheres, the resultant microsphere may exhibit prolonged imageability for as long as the microsphere remains intact (e.g., non-absorbed/degraded within the patient's body). Due to being integrally contained within the forming microsphere (e.g., as opposed to microsphere being coated or soaked in a contrasting solution), the imaging agent may be substantially resistant to being washed or otherwise removed from the resultant microspheres, allowing the microspheres to be tracked and monitored after administration during subsequent follow-up patient consultations as the microspheres remain visible under the different imaging procedures described. As the microspheres degrade/become absorbed within the patient's body, the imageability of the microsphere with likewise diminish. Such degradation of the microspheres may take several days before visibility of the microspheres becomes substantially reduced.

In some examples, the crosslinking reaction between OCMC and CCN may proceed under relatively benign conditions. For example, the crosslinking reaction may be carried out at ambient pressures and ambient temperatures (e.g., room temperature). In some examples, the reaction may be carried out at a temperature above ambient, such as, for example, 50° C. Exemplary ranges of temperatures in which the crosslinking reaction may be performed include between about 20° C. and about 70° C., and at about 50° C. In some examples, a lower reaction temperature may necessitate a longer reaction time to result in substantially similar diameter microspheres, or may result in smaller microspheres after a similar amount of time.

One advantage of performing the reaction at a temperature above room temperature may be the removal of water from the reaction mixture during the course of the reaction. For example, performing the crosslinking reaction at a temperature of about 50° C. may result in evaporation of water as the crosslinking reaction proceeds.

In some examples, the extent of crosslinking between molecules of the OCMC and CCN may affect mechanical properties of the resulting microspheres. For example, a greater crosslinking density generally may provide greater mechanical strength (e.g., fracture strain), while a lower crosslinking density may provide lower mechanical strength (e.g., fracture strain). The crosslinking density may also affect the degradation rate of the microsphere. For example, a greater crosslinking density may lead to a longer degradation time, while a lower crosslinking density may lead to a shorter degradation time. In some examples, the crosslink bonds may degrade through hydrolyzing of the C═N double bond.

As described above, the crosslinking reaction between OCMC and CCN is a modified emulsion-crosslinking reaction. In some examples, an emulsion-crosslinking reaction may be rate-limited by transport of the OCMC and CCN molecules, and may play a role in the reaction product (the crosslinked OCMC and CCN) being microspheres.

The size of the microspheres may be affected by reaction conditions, such as, for example, a stirring speed, a reaction temperature, a concentration of the OCMC and CCN molecules in the reaction emulsion, an amount of mixing of the emulsion, or a concentration of the surfactant in the emulsion. For example, increasing the concentration of each of the OCMC and CCN solutions from 1.5% w/v to 3% w/v while keeping the oxidation degree of OCMC at about 25% (about 25 oxidized repeating units per 100 total repeating units), the stirring speed at 600 revolutions per minute (rpm), the temperature at about 50° C., the reaction time at about 12 hours, and the amount of Span® 80 (sorbitane monooleate, available from Millipore Sigma, Saint Louis, Mo.) at about 0.3 mL/50 mL mineral oil, the average diameter of the microspheres may increase from about 600 μm to about 1100 μm. As another example, increasing the oxidation degree of OCMC from about 10% to about 25% while keeping the concentration of each of the OCMC and CCN solutions at about 1.5% w/v, the stirring speed at 600 rpm, the temperature at about 50° C., the reaction time at about 12 hours, and the amount of Span 80 at about 0.3 mL/50 mL mineral oil, the average diameter of the microspheres may increase from about 510 μm to about 600 μm.

In some examples, the reaction conditions may be selected to result in microspheres with a diameter between about 50 μm and about 2200 μm. In some examples, the reaction conditions may be selected to result in microspheres with a mean diameter of less than about 2000 μm, microspheres with a mean diameter of between about 100 μm and about 1200 μm, microspheres with a mean diameter of between about 100 μm and about 300 μm, microspheres with a mean diameter of between about 300 μm and about 500 μm, microspheres with a mean diameter of between about 500 μm and about 700 μm, microspheres with a mean diameter of between about 700 μm and about 900 μm, microspheres with a mean diameter of between about 900 μm and about 1200 μm, microspheres with a mean diameter of between about 1600 μm and about 2000 μm, microspheres with a mean diameter of less than about 300 μm, or microspheres with a mean diameter of greater than about 300 μm. In some examples, ethiodized oil(s) may be selected as the imaging agent for microspheres having a mean diameter of between about 300 μm to about 2200 μm, ethiodized oil(s) and radiopaque metal(s) may be selected as the imaging agent for microspheres having a mean diameter of between about 50 μm to about 300 μm, and SPIONs may be selected as the imaging agent for microspheres having a mean diameter of between about 50 μm to about 1200 μm.

In some examples, microspheres with different mean diameters may be used for different applications. For example, in some implementations, microspheres with a mean diameter between about 100 μm and about 300 μm may be loaded with a therapeutic agent (in addition to the imaging agent), such as a chemotherapeutic agent as described further below, and used to deliver the therapeutic agent to a therapy site, while also embolizing blood vessels with a diameter similar to the mean diameter of the microspheres. In some examples, microspheres with a mean diameter between about 300 μm and about 500 μm may be used similarly, and loaded with a therapeutic agent (in addition to the imaging agent). In some examples, microspheres with a larger mean diameter may be used as embolization materials, and may not be loaded with a therapeutic agent.

Once the reaction has proceeded for a desired amount of time to produce microspheres with a desired mean diameter, the water in the emulsion may be substantially fully removed during the crosslinking reaction. The microspheres may then be precipitated by a solvent, such as isopropanol. The oil/non-aqueous phase may then be removed, such as by decanting or centrifugation, and the microspheres may be washed (26). For example, the microspheres may be washed with hexane and acetone or Tween® 80 (polysorbate 80, available from Millipore Sigma, Saint Louis, Mo.). Finally, the microspheres may be dried (10) in air or under a vacuum.

In some examples, the crosslinking reaction may produce a plurality of microspheres with diameters distributed about a mean or median. In some cases, it may be advantageous to isolate microspheres with diameters within a smaller range or microspheres with substantially a single diameter. In some examples, the microspheres may be separated according to diameter by wet sieving in normal saline through a sieve or sieves with predetermined mesh size(s).

Any suitable amount of imaging agent(s) may be incorporated into the resultant microspheres. In some examples, the imaging agent(s) may account for about 0.5% to about 75% of the dry weight of the microsphere, such as between about 0.5% and about 5 wt. % of the dry weight of the microsphere for SPIONs, or between about 5% and about 75% of the dry weight of the microsphere for other imaging agents. The actual amount of imaging agent will depend on factors including, the type of imaging agent(s) used, the amount of CCN/CMC material incorporated into the aqueous mixtures, the total yield of imaging agent(s) integrally contained within the microspheres. In some examples, the imaging agent(s) may account for about 0.025% and about 20% of the final weight of the microsphere, such as between about 0.025% and about 0.5% of the final weight of the microsphere for SPIONs or between about 10% and about 20% of the final weight of the microsphere for other imaging agents.

The microspheres may be packaged for distribution in various ways. For example, the microspheres may be distributed as part of a kit. In some examples, the kit may include the microspheres disposed in a syringe or a vial. The kit may optionally include a catheter, a guide wire, and/or a container of solution in which the microspheres are to be suspended. The catheter may be used to inject the microspheres into a blood vessel of a patient. The guide wire may be used to position the catheter within the blood vessel.

In some examples, the kit may be an emergency trauma kit for acute embolization in massive bleeding trauma. Such a kit may include, for example, a syringe or vial and a plurality of microspheres disposed in the syringe or vial. In some examples, the microspheres may comprise an average diameter of between about 1600 μm and about 2000 μm. In other examples, the microspheres may comprise a different average diameter, such as an average diameter within a range listed in other portions of this application. In some examples, the kit may further include a catheter, a guide wire for positioning the catheter within a blood vessel, such as an artery, of the patient, and/or a container of solution in which the microspheres are to be suspended. Prior to injection of the microspheres, the solution may be aspirated into the syringe to form a suspension of the microspheres in the solution.

The microspheres may be used to embolize arteries to treat various conditions, including, for example, an arteriovenous malformation, a cerebral aneurysm, gastrointestinal bleeding, an epistaxis, primary post-partum hemorrhage, or the like. In some examples, the microspheres may be used for treating benign hypervascular tumors such as uterine fibroids. The improved visibility of the microspheres may provide a valuable tool to predict the clinical outcome and safety of a given procedure. The microspheres may also allow the user to detect the pattern of distribution and any non-target embolization, providing valuable information for the planning of future chemoembolization procedures as well as insight into the mechanisms of any potential complications with the disbursement of the microspheres. In addition, because the material of the microsphere is bioresorbable, the microspheres may provide a transient blood flow reduction, and favor restoration of artery integrity after embolization. Additionally, or alternatively, the resorbable microspheres can produce a limited hypoxia to reduce the stimulation of angiogenesis in tumors, which may be useful with regard to repeated arterial embolization for controlling tumor growth.

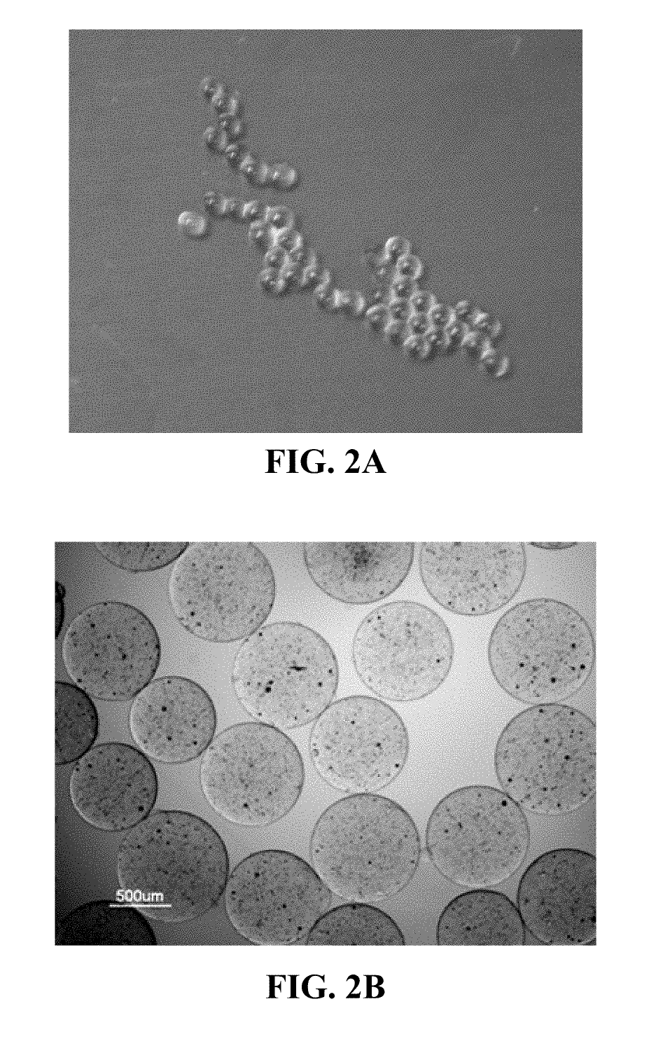

FIGS. 2A and 2B are a photograph and a light microscopy image of microspheres in accordance with one or more aspects of the disclosure. FIG. 2A illustrates that microspheres in accordance with the disclosure may be substantially spherical. FIG. 2B illustrates an example in which the diameter of the microspheres ranges from about 900 μm to about 1200 μm.

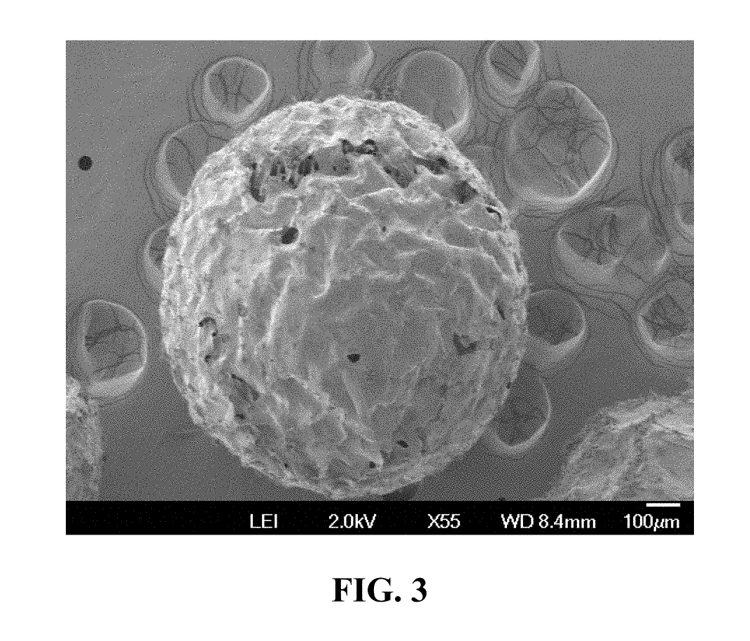

FIG. 3 is an example scanning electron microscopy (SEM) image of an exterior of a microsphere formed in accordance with aspects of this disclosure. Prior to collection of the SEM image, the microsphere was lyophilized. Before lyophilization (freeze drying), saline was removed from the microsphere by rinsing the microsphere repeatedly with deionized water. The resulting microsphere was frozen in liquid nitrogen, and lyophilized to remove any residual water from pores of the microsphere. The SEM image was obtained utilizing a JEOL JSM-6700 SEM (available from JEOL USA, Inc., Peabody, Mass.). FIG. 3 was collected at 55× magnification at 2.0 kilovolts (kV). The microsphere in FIG. 3 had a diameter of about 1100 μm.

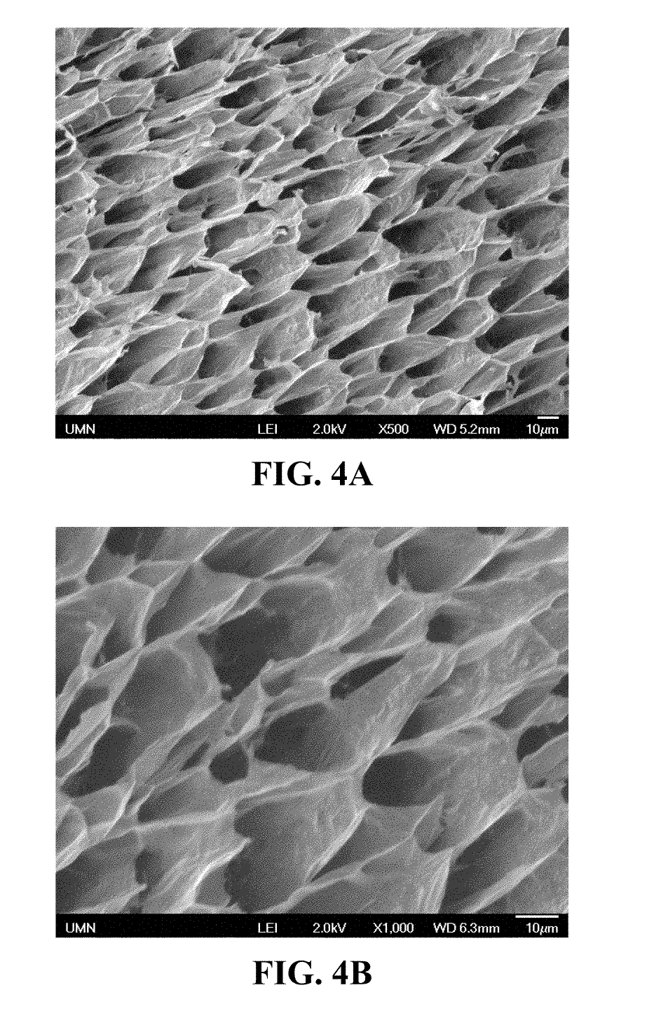

FIGS. 4A and 4B are examples of SEM images of a hydrogel prepared with CCN crosslinked with OCMC in accordance with aspects of the disclosure. The hydrogel was cut to expose an interior of the hydrogel and reveal the porous structure of the hydrogel. The SEM images were collected using a JEOL JSM-6700 SEM. FIG. 4A was collected at 500× magnification, while FIG. 4B was collected at 1000× magnification. Because the hydrogel was prepared using CCN crosslinked with OCMC, the internal structure of microspheres formed in accordance with this disclosure is expected to be similarly porous with the imaging agent integrally contained within the latticed structure.

In some examples, in addition to being utilized as an embolizing agent, the microspheres may be used to deliver a therapeutic agent to a therapy site. The microspheres comprising CCN crosslinked with OCMC may carry therapeutic agent due to functional groups on the CCN crosslinked with OCMC. For example, the microspheres may be loaded with a therapeutic agent, such as a chemotherapeutic agent, and used to deliver the chemotherapeutic agent to a tumor and/or to embolize arteries that feed the tumor. In some examples, the microspheres can be loaded with positively charged anti-cancer agents, such as doxorubicin, sunitinib, and irinotecan, etc., for malignant tumors, such as hepatocellular carcinoma and colorectal cancer. In other examples, the microspheres may be loaded with a cell, a bioactive molecule, or another drug. Additionally or alternatively, because the therapeutic agent will be localized at the same site post-delivery due to the microsphere, follow up imaging will provide information as to the extent of necrosis caused by the embolotherapy and may facilitate tumor regression analysis.

One example of a therapeutic agent that may be loaded into the microspheres is doxorubicin (available under the trade designation Adriamycin from Selleck Chemicals LLC, Houston, Tex., U.S.A.). Doxorubicin includes a protonated amino group and a plurality of hydroxyl groups, which may interact with functional groups, such as a carboxylic group, in the microsphere to bind to the microsphere via ionic interactions. While doxorubicin is provided as one example of a therapeutic agent which may be loaded into the microspheres of the present disclosure, other therapeutic agents may be used with the microspheres. For example, hydrophilic therapeutic agents may be utilized with the microspheres according to the disclosure. In particular, therapeutic agents that include at least one functional group that interacts with a carboxylic group, hydroxyl group or an aldehyde group are expected to be compatible with microspheres of the present disclosure. Examples of such therapeutic agents include idarubicin, sunitinib, epirubicin, irinotecan (available under the trade designation Camptosar® from Pfizer, New York, N.Y., U.S.A), ambroxol, and other therapeutic agents with at least one positively charged functional group.

In other examples, the therapeutic agent may be loaded onto the microspheres after formation of the microspheres. For example, the microspheres may be immersed in a solution of the therapeutic agent in a solvent to load the therapeutic agent onto the microsphere. In some examples, the therapeutic agent solution may have a concentration of between about 1 mg therapeutic agent per mL solvent (mg/mL) and about 25 mg/mL.

In some examples, the therapeutic agent may be loaded into the microspheres to a concentration of between about 0.3 mg therapeutic agent per mg dry microsphere (mg/mg) and about 0.75 mg/mg.

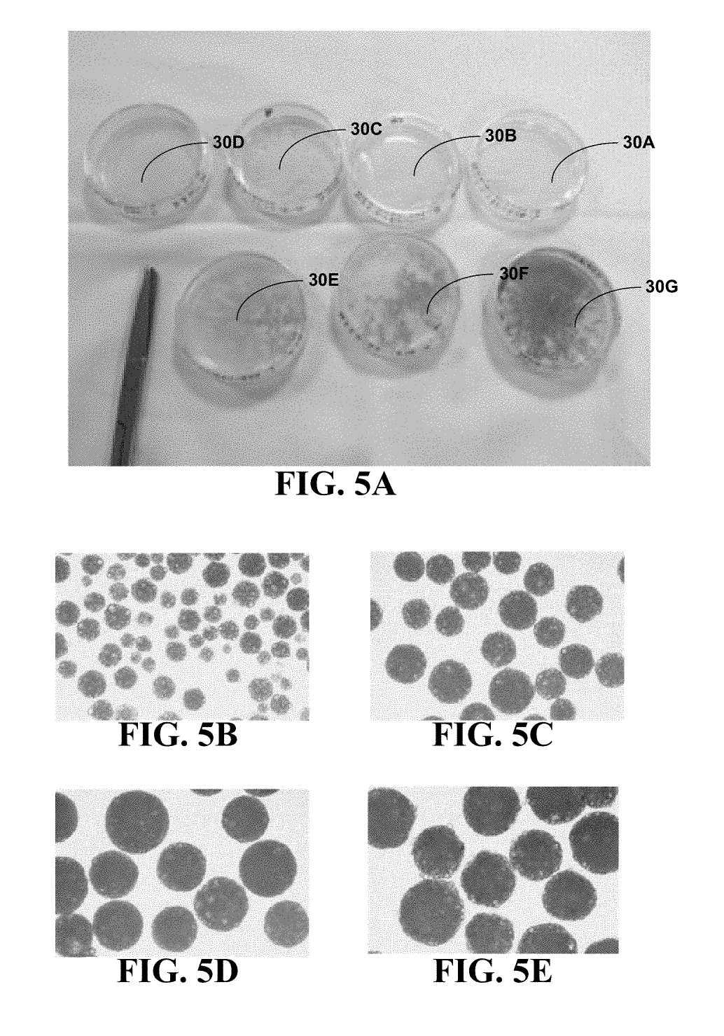

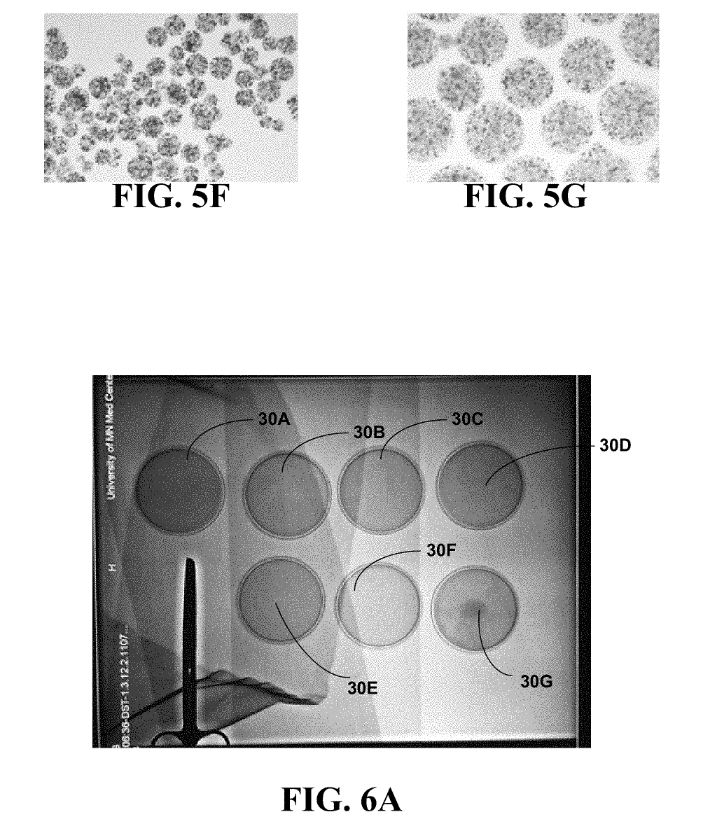

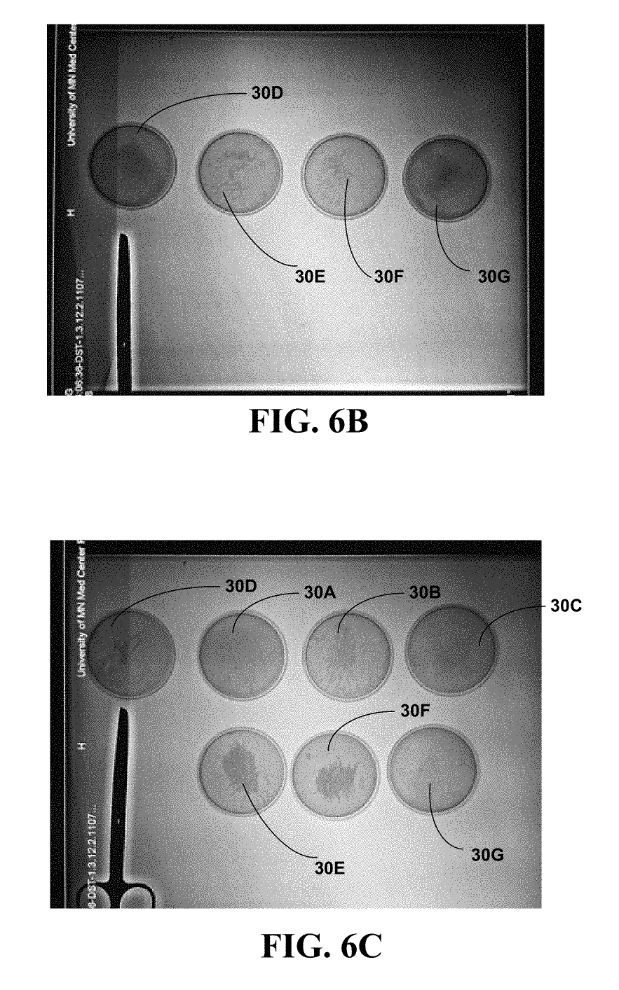

FIGS. 5A-5G and 6A-6C show a series of images taken of microspheres prepared in accordance with some of the examples described herein. Table 1 below provides a list of the components used to form microsphere samples 30A-30G along with the results of the X-ray contrast visibility of the microspheres of depending on the size of the microspheres.

TABLE 1

| | CCN/CMC | Ethiodized oil | | |
|---|---|---|---|---|
| Sample | (% w/v aqu. | (Lipiodol ®) | Tantalum | X-ray |
| No. | solv.) | (mL) | (g) | Contrast Visibility |
| 30A | 2.5 | 1 | 0 | Good Visibility for |
| | | | | sphere size >300 μm |
| 30B | 2.5 | 2 | 0 | Good Visibility for |
| | | | | sphere size >300 μm |
| 30C | 2.5 | 3 | 0 | Good Visibility for |
| | | | | sphere size >300 μm |
| 30D | 2.5 | 4 | 0 | Good Visibility for |
| | | | | sphere size >300 μm |
| 30E | 2.5 | 4 | 0.075 | Good Visibility for |
| | | | | sphere size >300 μm |
| 30F | 2.5 | 5 | 0.225 | Good Visibility for |
| | | | | sphere size >300 μm |
| 30G | 3.0 | 0 | 0.960 | Good Visibility for |
| | | | | all sphere sizes |

FIG. 5A is an image of microspheres 30A-30G formulated in accordance with Table 1. Microspheres 30A-30D were formed using only ethiodized oil as the imaging agent and had a generally white appearance. Microspheres 30E and 30F were formed using a combination of ethiodized oil and tantalum powder as the imaging agent and had a generally grey appearance. Microspheres 30G were formed using only tantalum powder as the imaging agent and had a generally grey/black appearance. The increase in the amount of tantalum content resulted in a generally darker appearance of the microsphere.

FIGS. 5B-5G are microscopic images taken of microsphere formed in accordance to the formulas of Table 1 of various diameter sizes of the microspheres under a magnification of 4×. FIG. 5B shows microspheres 30D with a microsphere diameter size of about 100 μm to about 300 μm. FIG. 5C shows microspheres 30D with a microsphere diameter size of about 300 μm to about 500 μm. FIG. 5D shows microspheres 30D with a microsphere diameter size of about 500 μm to about 700 μm. FIG. 5E shows microspheres 30E with a microsphere diameter size of about 500 μm to about 700 μm. FIG. 5F shows microspheres 30G with a microsphere diameter size of about 100 μm to about 300 μm. FIG. 5G shows microspheres 30G with a microsphere diameter size of about 500 μm to about 700 μm. Microspheres 30D (e.g., FIGS. 5B-5D) showed an uneven texture with bubble like oil pockets of the imaging agent with the larger sphere diameters (e.g., >300 μm) exhibiting a more spherical shape. Microspheres 30E (FIG. 5E) with an imaging agent formed of a combination of ethiodized oil and tantalum powder had an appearance similar to that of microspheres 30D (e.g., ethiodized oil only). Microspheres 30G (FIGS. 5F and 5G) with only tantalum powder as the imaging agent had a visibly darker appearance (e.g., to the human eye) and were substantially spherical in shape (e.g., spherical or nearly spherical).

FIGS. 6A-6C are fluoroscopic images taken of microspheres formed in accordance with the formulas of Table 1 of various diameter sizes of the microspheres. FIG. 6A shows microspheres 30A-30G under fluoroscopic x-ray with average diameter sizes of about 100 μm to about 300 μm. Microspheres 30G showed the best contrast visibility for diameter sizes less than 300 μm. FIG. 6B shows microspheres 30A-30G under fluoroscopic x-ray with average diameter sizes of about 300 μm to about 500 μm. All microspheres 30A-30G showed good contrast visibility with minimal difference between the different microspheres. FIG. 6C shows microspheres 30A-30G under fluoroscopic x-ray with average diameter sizes of about 500 μm to about 700 μm. For microsphere diameter greater than about 500 μm, an increase in the amount of ethiodized oil corresponded in an increase in visibility. The presence of tantalum powered also increased the visibility of the microspheres however, microspheres 30G containing only tantalum powder as the imaging agent did not exhibit the highest visibility. Microspheres 30D, 30E, and 30F exhibited the highest contrast visibility in the set.

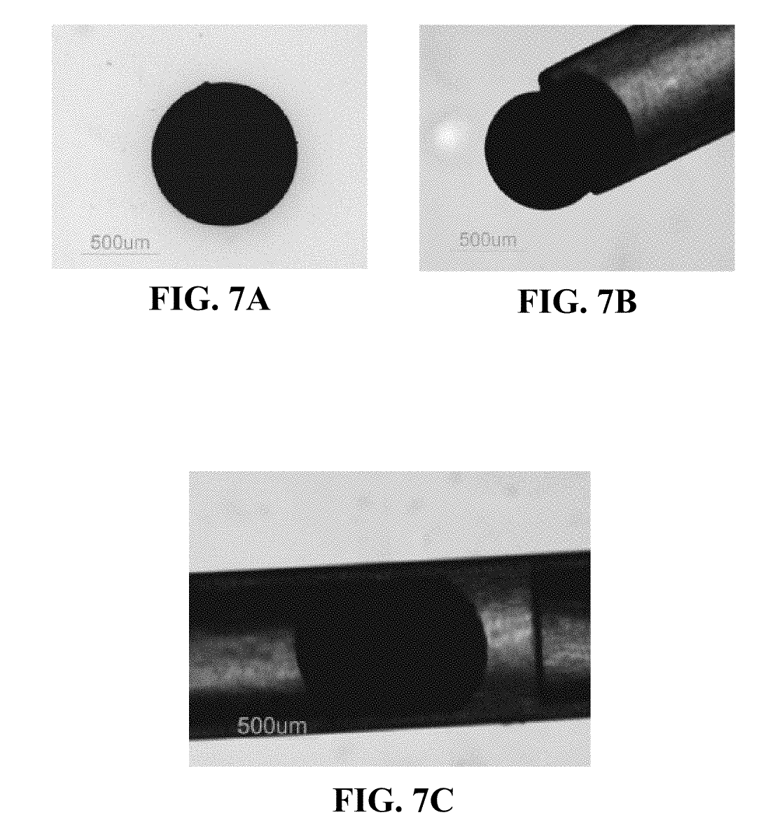

FIGS. 7A-7C are light microscopy images illustrating an example of the compressibility of a microsphere comprising CCN crosslinked with OCMC as the microsphere passes through a polyethylene tube. The microsphere has a diameter of about 925 μm and the catheter has an internal diameter of about 580 μm (PE-50). As FIGS. 7B and 7C illustrate, the microsphere can deform and pass through the internal cavity of the catheter.

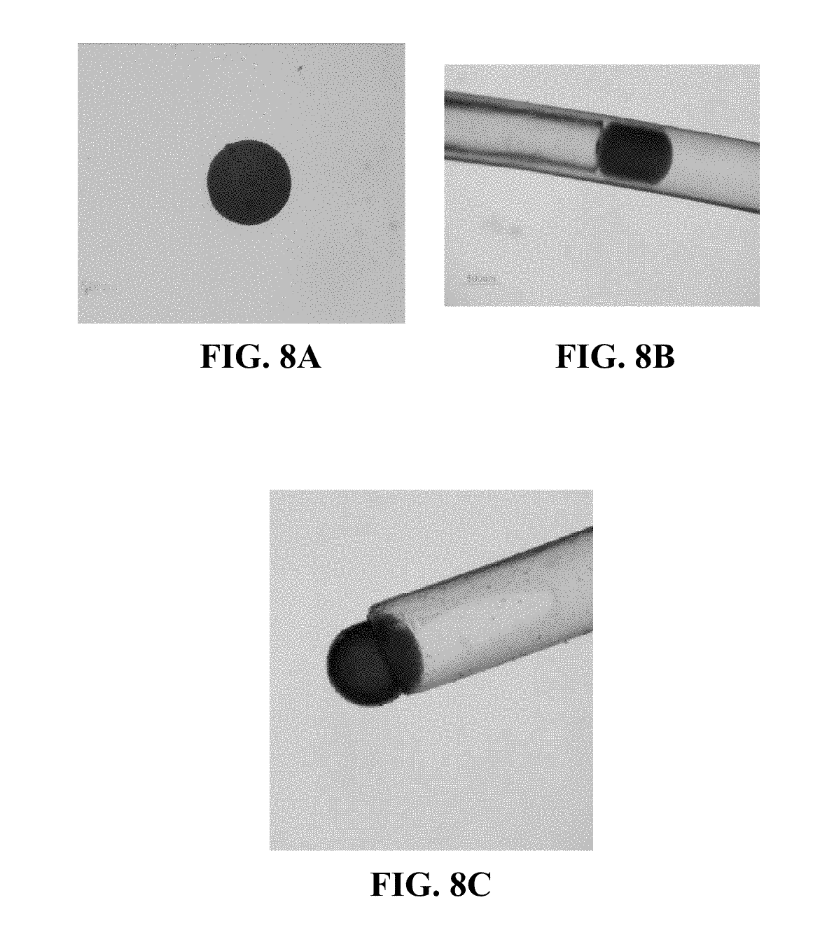

FIGS. 8A-8C are light microscopy images that illustrate another example of the compressibility of a microsphere comprising CCN crosslinked with OCMC and having a diameter of about 860 μm as the microsphere passes through a polyethylene tube. In FIGS. 8A-8C, the catheter again has an internal diameter of about 580 μm (PE-50). As FIGS. 8B and 8C illustrate, the microsphere can reversibly deform, pass through the internal cavity of the catheter, and return to a shape and size substantially similar to the shape and size of the microsphere before passing through the catheter.

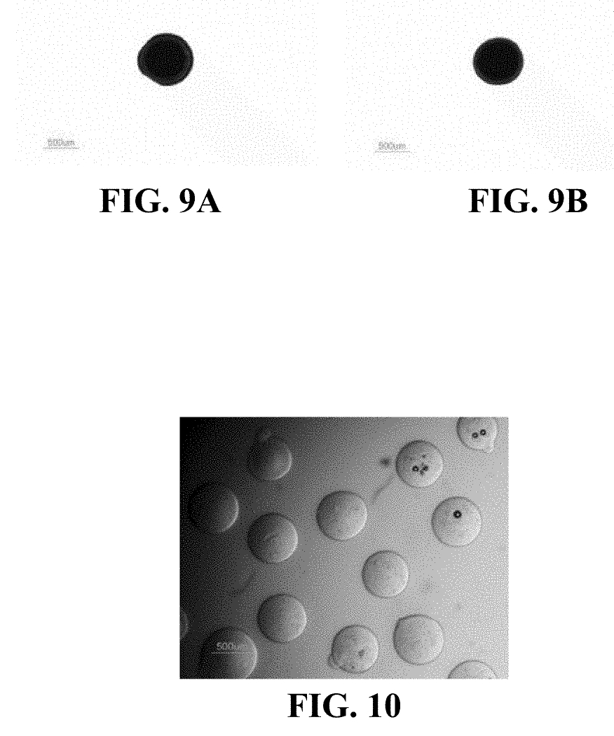

FIGS. 9A and 9B illustrate an example of the resiliency of a microsphere comprising CCN crosslinked with OCMC. The microsphere pictured in FIGS. 9A and 9B has a diameter of about 675 μm and was disposed in a polyethylene tube with an internal diameter of about 580 μm (PE-50) for about 24 hours prior to being released. The image shown in FIG. 9A was collected about 3 seconds after the microsphere was released from the PE tube, and the image shown in FIG. 9B was collected about 5 seconds after the microsphere was released. FIGS. 9A and 9B illustrate that the microsphere may recover its spherical shape and original size relatively quickly after being released from the PE tube.

FIG. 10 is a light microscopy image of an example of microspheres having diameters between about 500 μm and about 700 μm taken after the microspheres were injected through a catheter with an internal diameter of about 480 μm (2 French catheter, available from Boston Scientific Corp., Natick, Mass.). As illustrated in FIG. 10, the microspheres substantially retained their original, spherical shape.

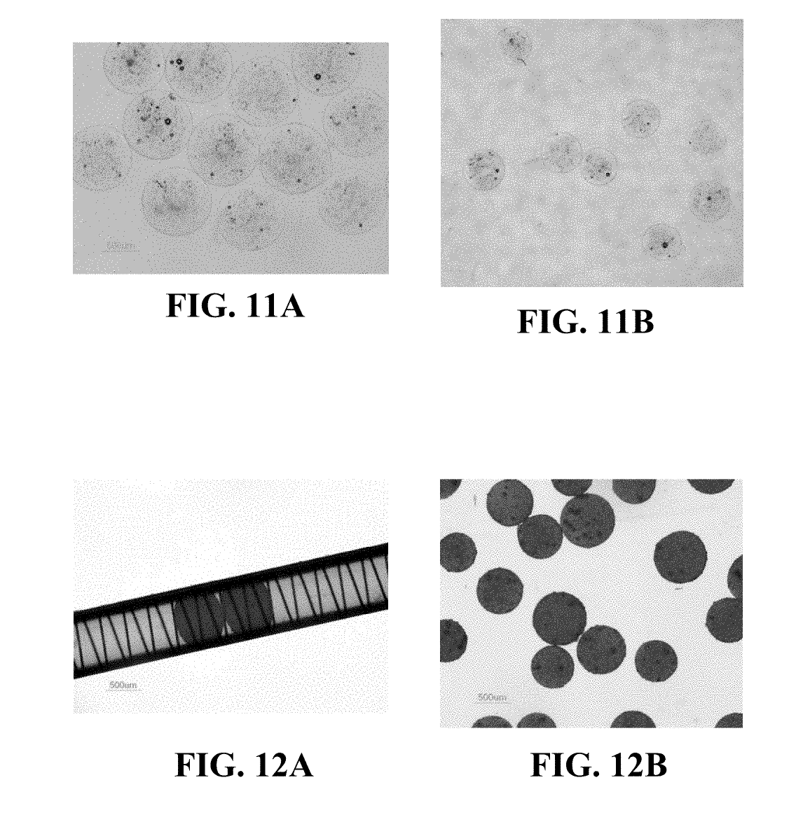

FIGS. 11A and 11B are light microscopy images of an example of microspheres having diameters between about 800 μm and about 1000 μm taken after the microspheres were injected through a catheter with an internal diameter of about 0.53 mm (2.3 French catheter, Terumo Medical Corp., Somerset, N.J.). As illustrated in FIGS. 11A and 11B, the microspheres substantially retained their original, spherical shape.

FIGS. 12A and 12B are light microscopy images of an example of microspheres loaded with doxorubicin while passing through a catheter and after passing through the catheter. The microspheres illustrated in FIGS. 12A and 12B have a diameter between about 500 μm and about 700 μm. The catheter shown in FIG. 12A had an internal diameter of about 0.53 mm (3 French catheter, Terumo Medical Corp., Somerset, N.J.). As shown in FIG. 12B, the microspheres substantially retained their original, spherical shape.

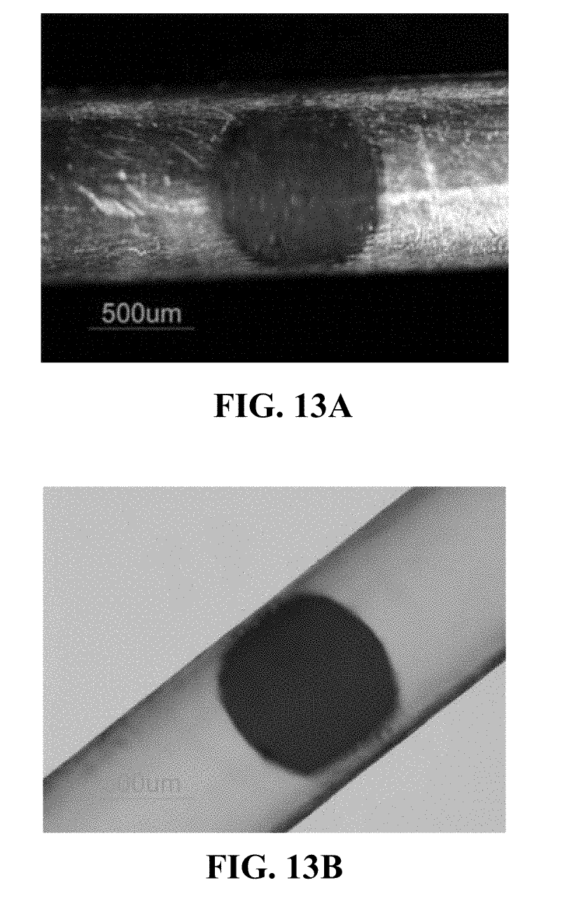

FIGS. 13A and 13B are light microscopy images of an example of microspheres loaded with doxorubicin while passing through a polyethylene tube. The microspheres illustrated in FIGS. 13A and 13B have a diameter between about 500 μm and about 700 μm. The catheter shown in FIGS. 13A and 13B had an internal diameter of about 580 μm (PE-50).

Microspheres according to the present invention may be suspended in a variety of solvents including, for example, saline, water, or the like. Microspheres comprising CCN crosslinked with OCMC may be somewhat stable when stored in water, but eventually may begin to degrade. FIG. 14 is a light microscopy image of an example of a plurality of microspheres after being stored for two months in water. The microspheres shown in FIG. 14 had a crosslinking density of about 10%. The microspheres shown in FIG. 14 have been dyed with Evan's blue to increase their visibility (e.g., by the human eye) in water. As FIG. 14 illustrates, the microspheres have begun to degrade and show decreased mechanical integrity.

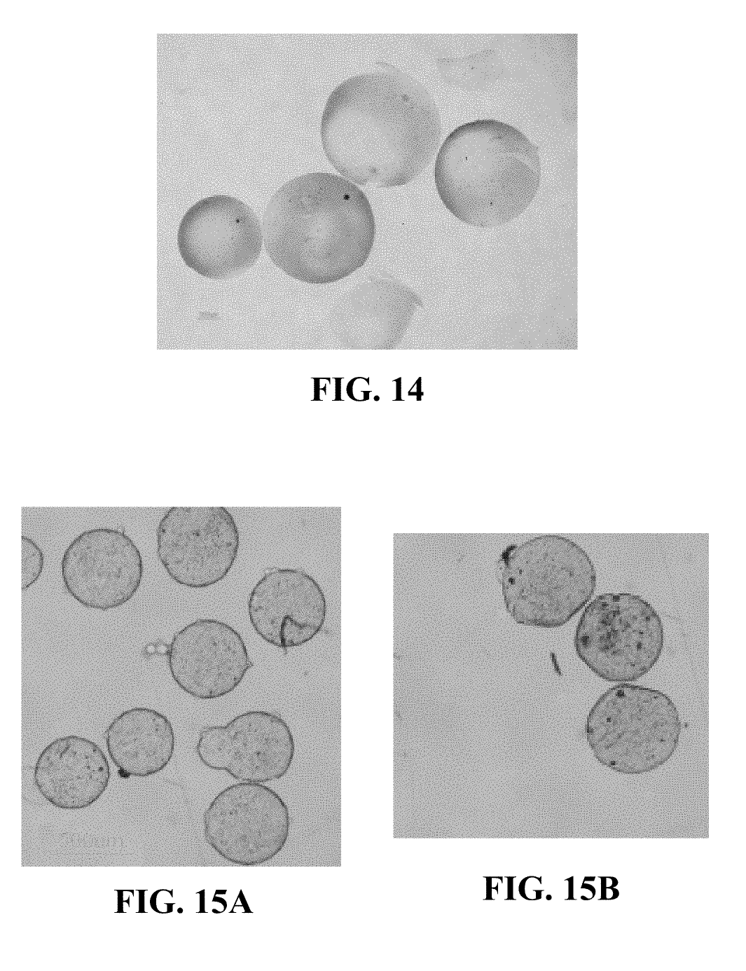

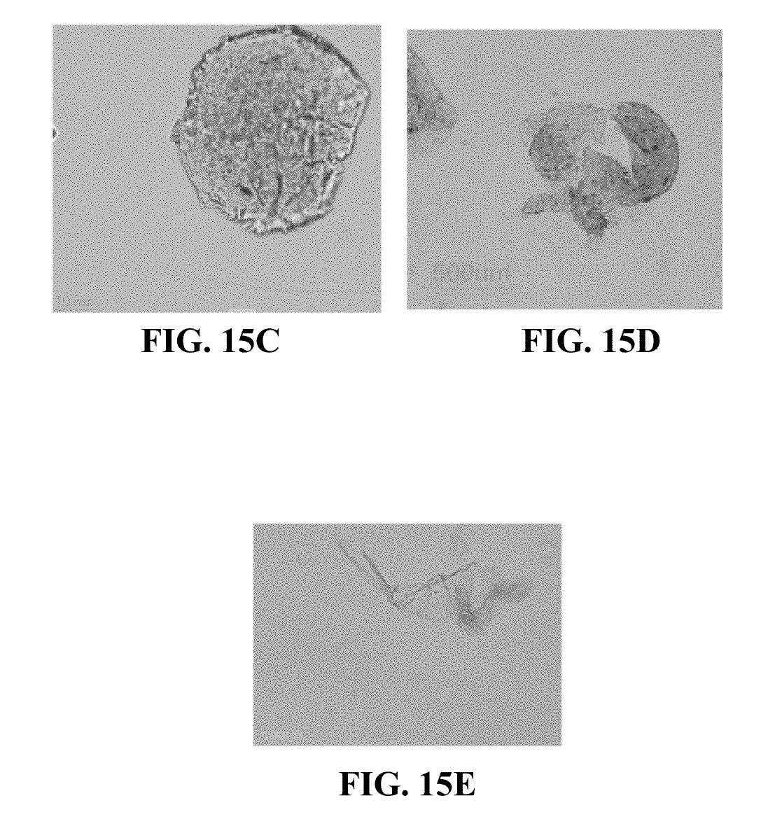

In some examples, microspheres comprising CCN crosslinked with OCMC may degrade more rapidly in the presence of an enzyme such as lysozyme. FIGS. 15A-15E are light microscopy images that illustrate an example of a plurality of microspheres degrading in the presence of lysozyme. The microspheres had a crosslinking density of about 10%. The medium surrounding the microspheres contained 4 mg/mL lysozyme and the microspheres and surrounding medium were kept at a temperature of about 37° C. for the duration of the test. FIG. 15A illustrates the appearance of the microspheres on day 0, soon after the microspheres were placed in the medium. FIG. 15B shows the appearance of the microspheres on day 3. FIG. 15C illustrates the appearance of a microsphere after 7 days. Visual evidence of the beginning of degradation is apparent. FIG. 15D shows the appearance of a microsphere on day 9, which shows degradation of the microsphere progressing, mechanical integrity decreasing, and the sphericity of the microsphere decreasing. Finally, FIG. 15E illustrates the appearance of a microsphere on day 14, at which time pieces of microsphere can be found in the medium, but the microsphere is no longer spherical.

As described above, the degradation time of the microspheres may be adjusted by increasing or decreasing the crosslink density in the microspheres. For example, a higher crosslink density, which may correspond to a higher oxidation degree of the OCMC, may lead to an increased degradation time, while a lower crosslink density (a lower oxidation degree of the OCMC) may lead to a decreased degradation time.

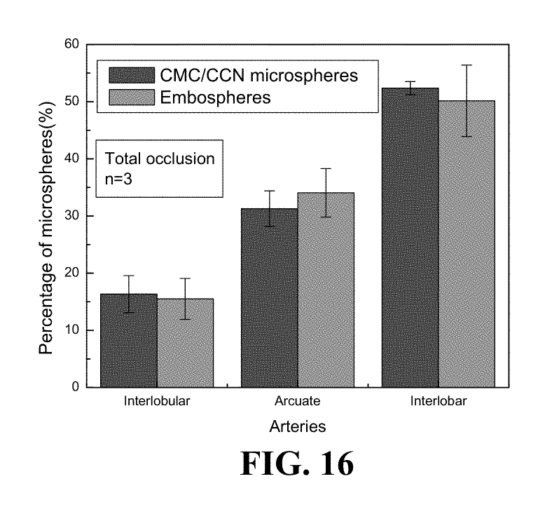

FIG. 16 is a bar graph that illustrates an example of the arterial distribution of the microspheres after embolization of three pairs of rabbit kidneys with microspheres having diameters between about 100 μm and about 300 μm. For comparative purposes, microspheres available under the trade designation Embosphere® (available from BioSphere Medical, Inc., Rockland, Mass., U.S.A.) having diameters between about 100 μm and about 300 μm were also used. In each example, microspheres were injected into the renal artery of live rabbits. The rabbits were then euthanized and the kidneys removed to assess the results of the embolization. The numbers of microspheres for each of the Embosphere® microspheres and the microspheres formed of CCN crosslinked with OCMC were counted at a first location in the interlobar artery (proximal to the injection site), the arcuate artery (median), and at a second location in the interlobar artery (distal to the injection site). The results are shown in FIG. 16 as a percentage of the microspheres counted at each location.

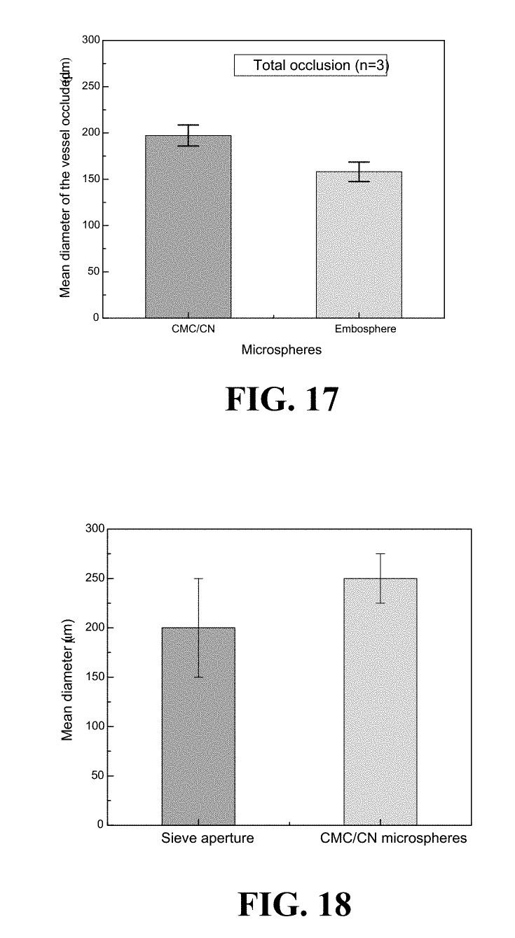

FIG. 17 is bar diagram that illustrates an example comparison between Embospheres® and microspheres formed of CCN crosslinked with OCMC of the mean diameter of the vessel occluded during an embolization procedure. In some examples, it may be desirable that the microspheres occlude arteries with a certain, predetermined diameter. Accordingly, it may be desirable to understand the relationship between a size range of the microspheres and the average diameter occluded by the microspheres. As in FIG. 16, the nominal diameter of the Embospheres® used in the example shown in FIG. 17 was between 100 μm and 300 μm. Similarly, the nominal diameter of the microspheres formed of CCN crosslinked with OCMC was between 100 μm and 300 μm. The mean diameter occluded by the Embospheres® was about 150 μm, while the mean diameter occluded by the microspheres formed of CCN crosslinked with OCMC was about 200 μm.

FIG. 18 is a bar diagram that illustrates an example determination of the mean diameter of the microspheres formed of CCN crosslinked with OCMC that were used in the embolization procedure that generated the results shown in FIG. 17. The bar labeled “CMC/CN microspheres” shows the mean diameter of the microspheres formed of CCN crosslinked with OCMC as determined by optical micrography. The bar labeled “Sieve aperture” shows the calibrated mean diameter of the microspheres formed of CCN crosslinked with OCMC, determined by passing sieves with different apertures and averaging the aperture sizes of the two adjacent sieves aperture, one of which the microspheres did not pass through and one of which the microspheres did pass through. In this way, the sieve aperture mean diameter may be considered the mean diameter of compressed microspheres formed of CCN crosslinked with OCMC. As shown in FIGS. 42 and 43, the sieve aperture mean diameter is substantially the same as the mean diameter of the vessel occluded by the microspheres formed of CCN crosslinked with OCMC. This suggests that determining a sieve aperture mean diameter for microspheres formed of CCN crosslinked with OCMC may predict a mean diameter of a vessel that may be occluded by the microspheres.

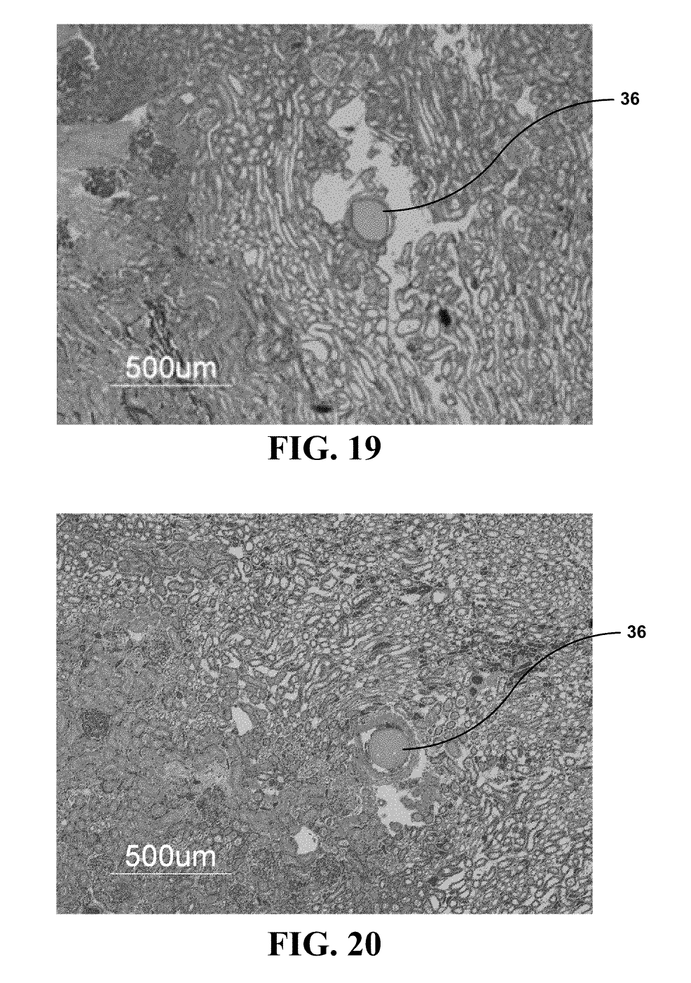

FIGS. 19 and 20 are example histology sections of kidney tissue showing arcuate artery in a kidney of a rabbit occluded with an embolic microsphere 36 according to aspects of the disclosure. As shown in FIGS. 19 and 20, the embolic microsphere 36 occludes substantially all of the artery. The small interval between the embolic microsphere 36 and the wall of the vessel is believed to be caused by sample processing as the microsphere 36 shrinks upon fixation.

Microspheres formed according to the present disclosure may be utilized for a number of applications. For example, one application for an embolic microsphere comprising CCN crosslinked by OCMC is transarterial chemoembolization (TACE) of liver tumors. TACE for unresectable hepatocellular carcinoma (HCC) is an approved treatment modality that increases patient survival compared to intravenous chemotherapy. TACE includes intraarterial (via the hepatic artery) injection of chemotherapeutic agents followed by embolization of tumoral feeding arteries. The trend in TACE is to use drug eluting beads loaded with chemotherapeutic agents that are progressively released into the tumor. Drug eluting TACE is associated with less systemic toxicity and a better patient tolerance. Because the microsphere comprising CCN crosslinked by OCMC is bioresorbable and is thus absorbed by the body of the patient over time after injection, the release profile of the chemotherapeutic agents may be controlled. Additionally, the microsphere comprising CCN crosslinked by OCMC may act as combination chemotherapeutic agent carriers and embolization agents. Furthermore, because the microsphere comprising CCN crosslinked by OCMC are bioresorbable, artery integrity may be restored upon resorption, which may be advantageous in some examples.

Another application for microspheres comprising CCN crosslinked by OCMC is Uterine Fibroids Embolization (UFE). Uterine Fibroids are benign muscular tumors that grow in the wall of the uterus. Uterine fibroids can grow as a single tumor or as many tumors. Uterine fibroids can be either as small as an apple seed or as big as a grapefruit. In unusual cases uterine fibroids can become very large. An increasingly accepted therapy technique for uterine fibroids is UFE. The main purpose of UFE is to reduce the size of the fibroid and to treat excessive uterine bleeding. In essence, UFE involves the placement of a catheter into the uterine arteries and injection of embolization microspheres into the uterine arteries to achieve fibroid devascularization and progressive shrinkage. Use of bioresorbable microspheres comprising CCN crosslinked by OCMC may facilitate restoration of uterine artery integrity after embolization.

### EXAMPLES

### Example 1: Preparation of OCMC

Approximately 1 g of sodium carboxymethyl cellulose (Sigma-Aldrich, St. Louis, Mo., M_{w} approximately 700,000 g/mol) and 80 mL distilled water were added to a 250-mL flask. After the carboxymethyl cellulose dissolved substantially completely, 25% molar equivalent of sodium periodate in 20 mL distilled water was added to the flask. The reaction was allowed to proceed for 24 hours at approximately 25° C. After 24 hours, approximately 0.21 g ethylene glycol was added to the flask to stop the reaction. After an additional 30 minutes, the mixture was poured into a dialysis tube (MWCO 3500) to dialyze against distilled water for 3 days. Dry product was obtained by lyophilizing the dialyzed solution. The resulting OCMC was labeled OCMC-II.

### Example 2: Preparation of OCMC

Approximately 1 g of sodium carboxymethyl cellulose (Sigma-Aldrich, St. Louis, Mo., M_{w} approximately 700,000 g/mol) and 80 mL distilled water were added to a 250-mL flask. After the carboxymethyl cellulose dissolved substantially completely, 10% molar equivalent of sodium periodate in 20 mL distilled water was added to the flask. The reaction was allowed to proceed for 24 hours at approximately 25° C. After 24 hours, approximately 0.08 g ethylene glycol was added to the flask to stop the reaction. After an additional 30 minutes, the mixture was poured into a dialysis tube (MWCO 3500) to dialyze against distilled water for 3 days. Dry product was obtained by lyophilizing the dialyzed solution. The resulting OCMC was labeled OCMC-I.

### Example 3: Preparation of OCMC

Approximately 1 g of sodium carboxymethyl cellulose (Sigma-Aldrich, St. Louis, Mo., M_{w} approximately 700,000 g/mol) and 80 mL distilled water were added to a 250-mL flask. After the carboxymethyl cellulose dissolved substantially completely, 50% molar equivalent of sodium periodate in 20 mL distilled water was added to the flask. The reaction was allowed to proceed for 24 hours at approximately 25° C. After 24 hours, approximately 0.42 g ethylene glycol was added to the flask to stop the reaction. After an additional 30 minutes, the mixture was poured into a dialysis tube (MWCO 3500) to dialyze against distilled water for 3 days. Dry product was obtained by lyophilizing the dialyzed solution. The resulting OCMC was labeled OCMC-III.

### Example 4: Preparation of CCN

In a 3-neck flask, approximately 2 g chitosan (Sigma-Aldrich, St. Louis, Mo., greater than 75% deacetylated) was added to a mixture of approximately 16 g sodium hydroxide, approximately 20 mL distilled water, and approximately 20 mL isopropanol. The mixture was stirred at approximately 25° C. for approximately 24 hours. Before carboxymethylation, the flask was maintained in a water bath at approximately 50° C. for approximately 1 hour. Approximately 16 g monochloroacetic acid (Sigma-Aldrich, St. Louis, Mo.) in 10 mL isopropanol then was added dropwise into the reaction mixture. The reaction mixture was stirred at approximately 50° C. for an additional 4 hours, and the reaction was stopped by adding approximately 80 mL of 70% ethanol. The precipitate was filtered and rinsed thoroughly with 70-90% ethanol and vacuum dried at room temperature.

The dried product was dissolved in approximately 100 mL water and homogenized for 2 hours. Any insoluble residue present in the mixture was removed by centrifuging. The supernatant was dialyzed in an MWCO 3500 dialysis tube against distilled water and then lyophilized. The resulting CCN was labeled CCN-I.

### Example 5: Preparation of CCN

In a 3-neck flask, approximately 2 g chitosan (Sigma-Aldrich, St. Louis, Mo., greater than 75% deacetylated) was added to a mixture of approximately 8 g sodium hydroxide, approximately 10 mL distilled water, and approximately 10 mL isopropanol. The mixture was stirred at room temperature for approximately 24 hours. Before carboxymethylation, the flask was maintained in a water bath at approximately 50° C. for approximately 1 hour. Approximately 8 g monochloroacetic acid (Sigma-Aldrich, St. Louis, Mo.) in 5 mL isopropanol then was added dropwise into the reaction mixture. The reaction mixture was stirred at approximately 50° C. for an additional 4 hours, and the reaction was stopped by adding approximately 80 mL of 70% ethanol. The precipitate was filtered and rinsed thoroughly with 70-90% ethanol and vacuum dried at room temperature.

The dried product was dissolved in approximately 100 mL water and homogenized for 2 hours. Any insoluble residue present in the mixture was removed by centrifuging. The supernatant was dialyzed in an MWCO 3500 dialysis tube against distilled water and then lyophilized. The resulting CCN was labeled CCN-II.

### Example 6: Preparation of CCN and OCMC Microspheres without Imaging Agent(s)

Approximately 0.075 g of CCN-I was mixed in approximately 5 mL of water to form a 1.5% w/v CCN-I solution. Similarly, approximately 0.075 g OCMC-I was mixed in approximately 5 ml water to form a 1.5% w/v OCMC-I solution. The CCN-I and OCMC-I solutions were then mixed. The mixture was added to approximately 50 mL mineral oil containing between 0.2 mL and 0.5 mL sorbitane monooleate to form an emulsion. The emulsion was homogenized for approximately 45 minutes. The aqueous phase of the emulsion was allowed to evaporate over night at approximately 45° C. with constant stirring. The crosslinked CCN and OCMC was isolated by precipitation in isopropanol followed by centrifugation to remove the oil phase. The resulting microspheres were washed thoroughly in acetone before being dried under vacuum. The mean diameter of the microspheres, measured in normal saline by a light microscope, was approximately 515±3 μm.

### Example 7: Preparation of CCN and OCMC Microspheres without Imaging Agent(s)

Approximately 0.075 g of CCN-I was mixed in approximately 5 mL of water to form a 1.5% w/v CCN-I solution. Similarly, approximately 0.075 g OCMC-II was mixed in approximately 5 ml water to form a 1.5% w/v OCMC-I solution. The CCN-I and OCMC-II solutions were then mixed. The mixture was added to approximately 50 mL mineral oil containing between 0.2 mL and 0.5 mL sorbitane monooleate to form an emulsion. The emulsion was homogenized for approximately 45 minutes. The aqueous phase of the emulsion was allowed to evaporate over night at approximately 45° C. with constant stirring. The crosslinked CCN and OCMC was isolated by precipitation in isopropanol followed by centrifugation to remove the oil phase. The resulting microspheres were washed thoroughly in acetone before being dried under vacuum. The mean diameter of the microspheres, measured in normal saline by a light microscope, was approximately 594±3

### Example 8: Preparation of CCN and OCMC Microspheres without Imaging Agent(s)

Approximately 0.075 g of CCN-I was mixed in approximately 5 mL of water to form a 1.5% w/v CCN-I solution. Similarly, approximately 0.075 g OCMC-III was mixed in approximately 5 ml water to form a 1.5% w/v OCMC-I solution. The CCN-I and OCMC-III solutions were then mixed. The mixture was added to approximately 50 mL mineral oil containing between 0.2 mL and 0.5 mL sorbitane monooleate to form an emulsion. The emulsion was homogenized for approximately 45 minutes. The aqueous phase of the emulsion was allowed to evaporate over night at approximately 45° C. with constant stirring. The crosslinked CCN and OCMC was isolated by precipitation in isopropanol followed by centrifugation to remove the oil phase. The resulting microspheres were washed thoroughly in acetone before being dried under vacuum. The mean diameter of the microspheres, measured in normal saline by a light microscope was approximately 702±3 μm.

### Example 9: Preparation of CCN and OCMC Microspheres without Imaging Agent(s)

Approximately 0.1 g of CCN-II was mixed in approximately 5 mL of water to form a 2% w/v CCN-I solution. Similarly, approximately 0.1 g OCMC-II or 0.1 OCMC-III was mixed in approximately 5 ml water to form a 2% w/v OCMC-II solution or a 2% w/v OCMC-III solution. The CCN-I and OCMC-I solutions were then mixed. The mixture was added to approximately 50 mL mineral oil containing between 0.2 mL and 0.5 mL sorbitane monooleate to form an emulsion. The emulsion was homogenized for approximately 45 minutes. The aqueous phase of the emulsion was allowed to evaporate over night at approximately 45° C. with constant stirring. The crosslinked CCN and OCMC was isolated by precipitation in isopropanol followed by centrifugation to remove the oil phase. The resulting microspheres were washed thoroughly in acetone before being dried under vacuum. The mean diameter of the microspheres, measured in normal saline by a light microscope was approximately 2000 μm.

### Example 10: Preparation of Doxorubicin-Loaded Microspheres without Imaging Agent(s)

Microspheres disposed in saline and having wet weight of about 150 mg were added into a 22-mL glass vial. (The microspheres had a dry weight of about 17 mg and were formed from OCMC-II and CCN-III.) Excess saline was removed with a pipette. About 20 mL doxorubicin solution (about 2 mg doxorubicin/mL solution) was formed by dissolving doxorubicin in a saline/hydrochlric acid solution having a pH between about 2.5 and about 4.5 and was added into the vial. An amount of doxorubicin remaining in the loading solution after loading of the microspheres was determined by measuring the absorbance at 482 nm using a Beckman UV-Visible spectrophotometer and comparison to a standard curve constructed from solutions of known concentrations of drug. The maximum loading is between about 0.3 and about 0.7 mg doxorubicin per mg dry microspheres, depending on the size of the microspheres.

### Example 11: Preparation of CCN and OCMC Microspheres with Imaging Agent(s)

About 0.125 g of CCN was mixed in about 5 mL of water in a 20-mL glass vial to form a 2.5% w/v CCN solution. About 5 mL of Lipiodol® ethiodized oil was mixed with the 5 mL CCN solution. Similarly, about 0.125 g OCMC was mixed in about 5 ml water to form a 2.5% w/v OCMC solution. The CCN and OCMC solutions including the ethiodized oil were then mixed. The final mixture will then be added into a 50-mL mineral oil containing 0.1 mL Span® 80 under stirring at 600 rpm at room temperature. After about 15 min, the temperature was increased to 37° C. using a water bath. After 1 hr, an additional 0.6 mL Span® 80 was added to the mixture and the temperature increased to 45° C. After about sixteen hours later, the water bath was removed and the microspheres stirred for 30 min at room temperature to allow the mixture to cool to room temperature. The oil layer was then decanted as much as possible and the resultant microspheres rinsed with about 15 mL 5% Tween® 80 saline solution (polysorbate 80, available from Millipore Sigma, Saint Louis, Mo.) followed by about 50 mL saline 3 times; repeating the rinsing process once. The final microspheres were stored in saline at 2-8° C.

### Example 12: Preparation of CCN and OCMC Microspheres with Imaging Agent(s)

About 0.125 g of CCN was mixed in about 5 mL of water in a 20-mL glass vial to form a 2.5% w/v CCN solution. About 5 mL of Lipiodol ethiodized oil was mixed with about 0.225 g tantalum. The ethiodized oil and tantalum were then added to the 5 mL CCN solution. Similarly, about 0.125 g OCMC was mixed in about 5 ml water to form a 2.5% w/v OCMC solution. The CCN and OCMC solutions including the ethiodized oil were then mixed. The final mixture will then be added into a 50-mL mineral oil containing 0.1 mL Span® 80 under stirring at 600 rpm at room temperature. After about 15 min, the temperature was increased to 37° C. using a water bath. After 1 hr, an additional 0.6 mL Span® 80 was added to the mixture and the temperature increased to 45° C. After about sixteen hours later, the water bath was removed and the microspheres stirred for 30 min at room temperature to allow the mixture to cool to room temperature. The oil layer was then decanted as much as possible and the resultant microspheres rinsed with about 15 mL 5% Tween® 80 saline solution followed by about 50 mL saline 3 times; repeating the rinsing process once. The final microspheres were stored in saline at 2-8° C.

### Example 13: Preparation of CCN and OCMC Microspheres with Imaging Agent(s)

About 0.15 g of CCN was mixed in about 5 mL of water in a 20 mL glass vial to form a 3% w/v CCN solution. About 0.96 g tantalum powder was then added to the 5 mL CCN solution. Similarly, about 0.15 g OCMC was mixed in about 5 ml water to form a 3% w/v OCMC solution. The CCN and OCMC solutions including the ethiodized oil were then mixed. The final mixture will then be added into a 50 mL mineral oil containing 0.1 mL Span® 80 under stirring at 600 rpm at room temperature. After about 15 min, the temperature was increased to 37° C. using a water bath. After 1 hr, an additional 0.6 mL Span® 80 was added to the mixture and the temperature increased to 45° C. After about sixteen hours later, the water bath was removed and the microspheres stirred for 30 min at room temperature to allow the mixture to cool to room temperature. The oil layer was then decanted as much as possible and the resultant microspheres rinsed with about 15 mL 5% Tween® 80 saline solution followed by about 50 mL saline 3 times; repeating the rinsing process once. The final microspheres were stored in saline at 2-8° C.

### Example 14: Synthesis of MRI Visible Microspheres

About 5 mL of a 4% w/v CCN (1.45 ratio of x monomers toy monomers) aqueous solution and either 0.2 mL or 0.4 mL of super paramagnetic iron oxides (SPION) (a ferumoxides injectable solution available under the trade designation Feridex I.V. from Berlex Laboratories, Inc., Whippany, N.J.) were mixed well in a 20-mL glass vial. About 5 mL of 4% w/v OCMC (0.25 degree of oxidation) aqueous solution was added to the mixture and mixed well. The final mixture was then added into 50 mL of mineral oil containing about 0.1 mL of Span® 80 under stirring at 500 rpm. After about 15 min, the temperature was increased to about 37° C. After about 1 hour, about 0.6 mL of Span® 80 was added, and the temperature increased to about 45° C. for overnight reaction. The MRI visible microspheres had diameters between about 100 μm and about 1200 μm.

### Example 15: Light Microscopy of MRI Visible Microspheres

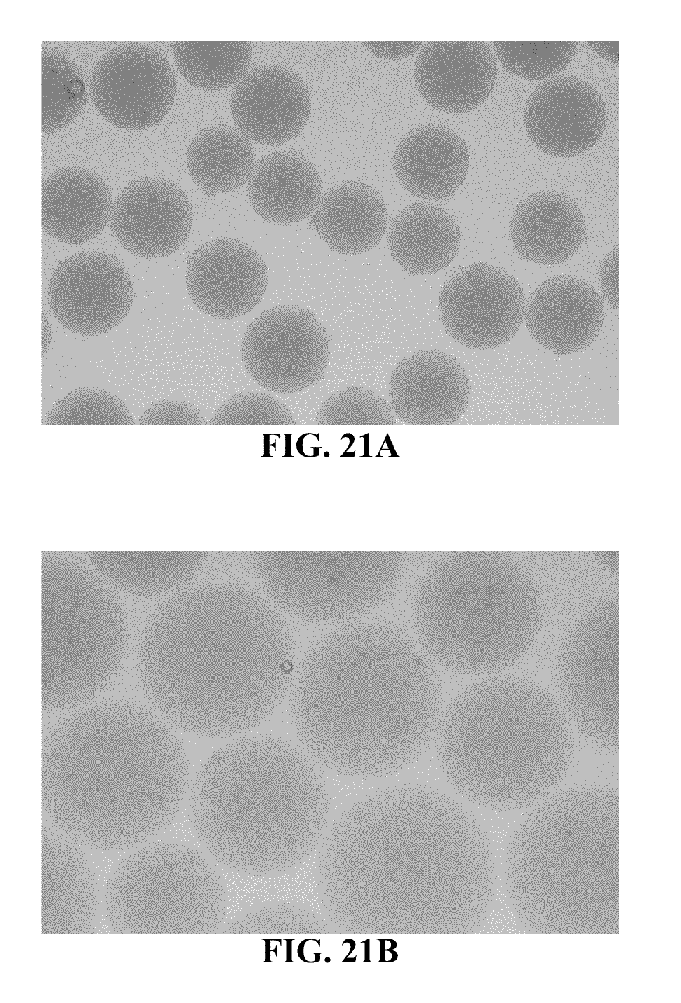

The MRI visible microspheres prepared per the technique of Example 14 were separated by size and imaged using a light microscope. FIG. 21A is a light microscopy image illustrating the appearance of MRI visible microspheres having diameters between about 300 μm and about 500 μm. FIG. 21B is a light microscopy image illustrating the appearance of MM visible microspheres having diameters between about 700 μm and about 900 μm. The MRI visible microspheres were substantially round and brown-yellow in color due to the SPIONs. In general, the higher the concentration of the SPIONs in the microspheres, the darker the microspheres.

### Example 16: Confocal Microscopy of MRI Visible Microspheres

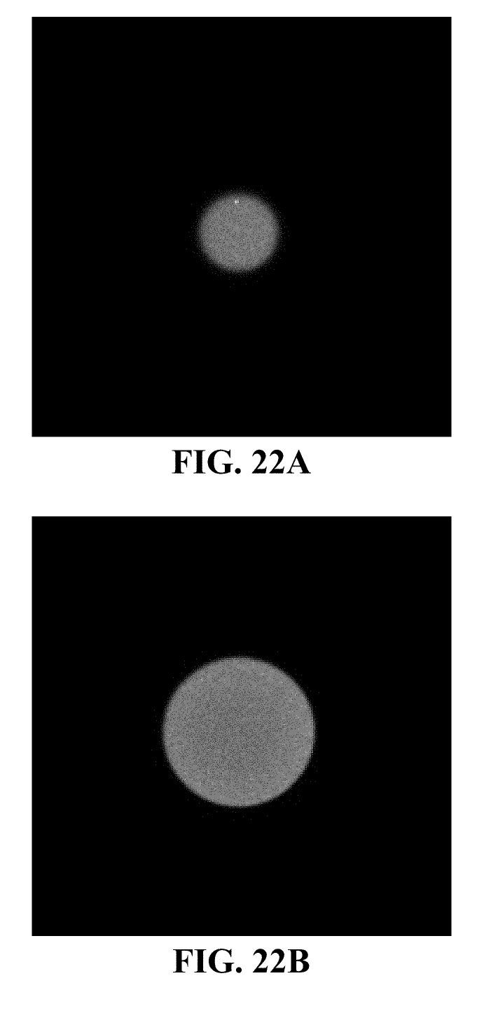

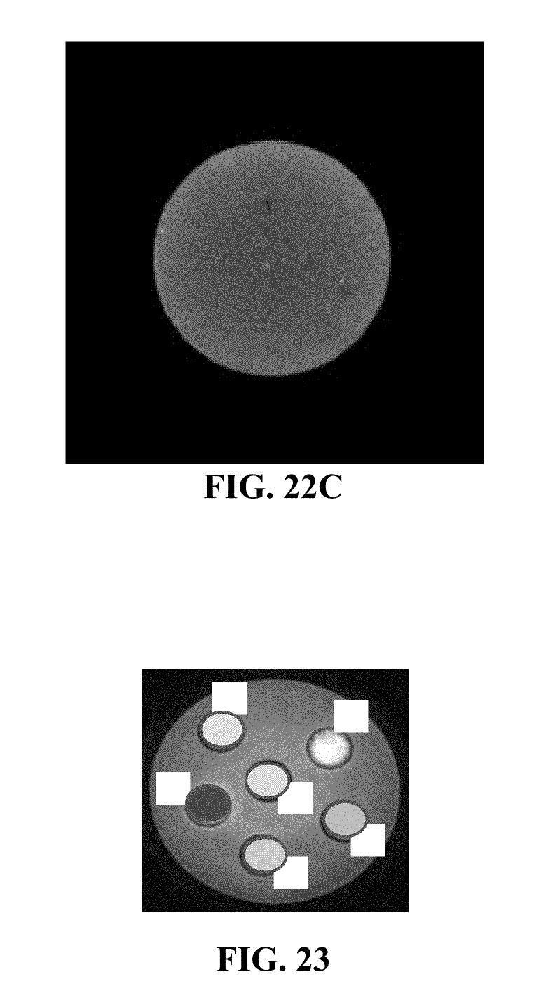

Multiphoton confocal microscopy was used to evaluate the homogeneity of a MRI visible microsphere prepared by the technique of Example 14 by scanning the microspheres layer by layer along the Z axis and then assembling the 2D images into a 3D image. FIGS. 22A-22C are fluorescent confocal microscopy images of the MM visible microsphere at a top focal plane, a middle focal plane, and an equatorial focal plane, respectively. As shown in FIGS. 22A-22C, the microsphere fluoresced evenly throughout the entire microsphere. This even fluorescence indicates an even distribution of the SPIONs inside the microsphere.

### Example 17: MRI Imaging of MRI Visible Microspheres

MRI visible microspheres were prepared according to the technique of Example 14, but with concentrations of SPIONs shown in Table 2. The MRI visible microspheres were placed in 2% agar gel in NMR tubes in six samples. The Fe concentration in agar was calculated by diving the SPION amount in the beads that were put into the agar gel by the total volume of the agar gel. The samples were arranged as shown in FIG. 23.

TABLE 2

| | Bead formulation | Fe concentration in Agar |
|---|---|---|
| Sample | (mg Fe/mL) | (mg/mL) |
| 1 | 3.73 | 0.5 |
| 2 | 3.73 | 1 |
| 3 | 3.73 | 2 |
| 4 | 2.22 | 0.5 |
| 5 | 2.22 | 1 |
| 6 | 0 | 0 |

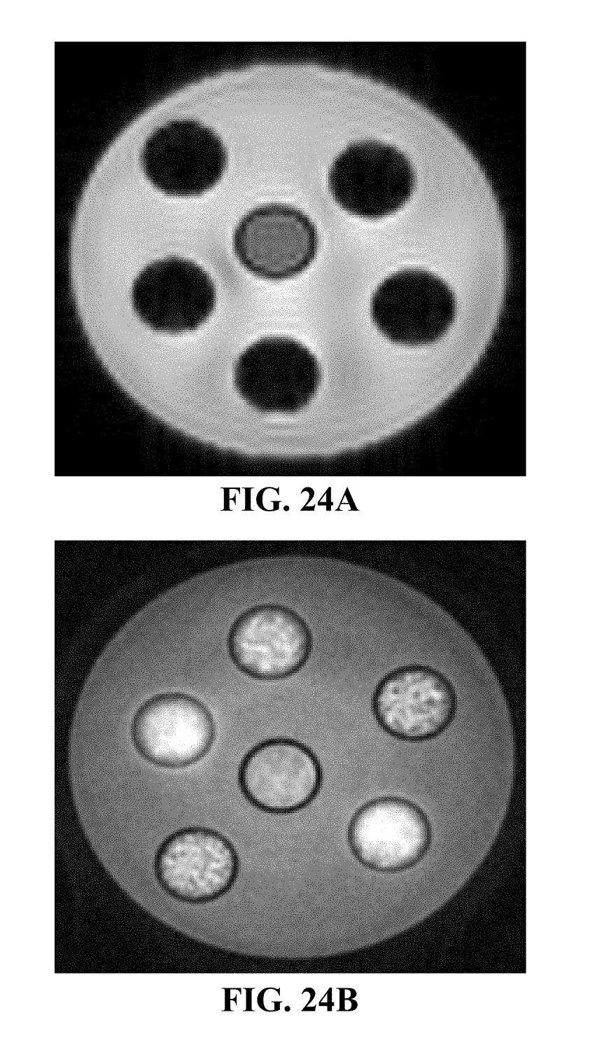

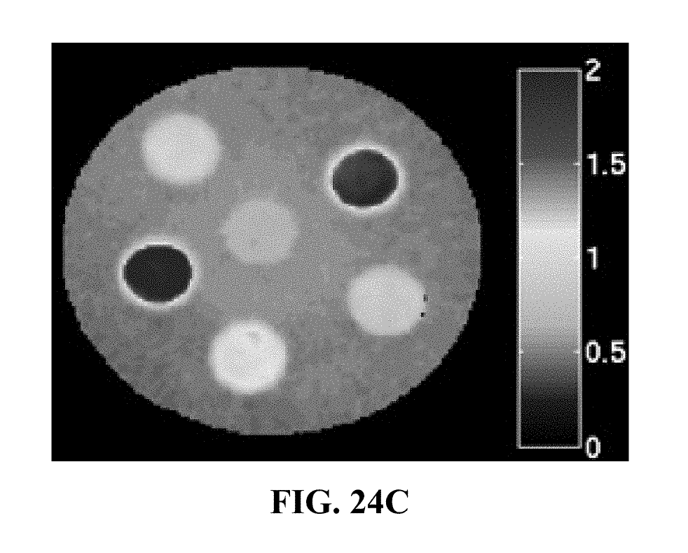

FIGS. 24A-24C are MM images of MRI visible microsphere samples collected using different MRI imaging techniques. The image shown in FIG. 24A was collected using conventional gradient recalled echo (GRE) imaging. As shown in FIG. 24A, all samples except the control (sample 6) imaged as dark spots due to the ultrashort T*2 caused by the SPIONs. The image shown in FIG. 24B was collected using multiband sweep imaging with Fourier transformation (MB-SWIFT), and shows greater T₁ positive contrast between the samples. This is confirmed by the MB-SWIFT relaxivity image shown in FIG. 24C.

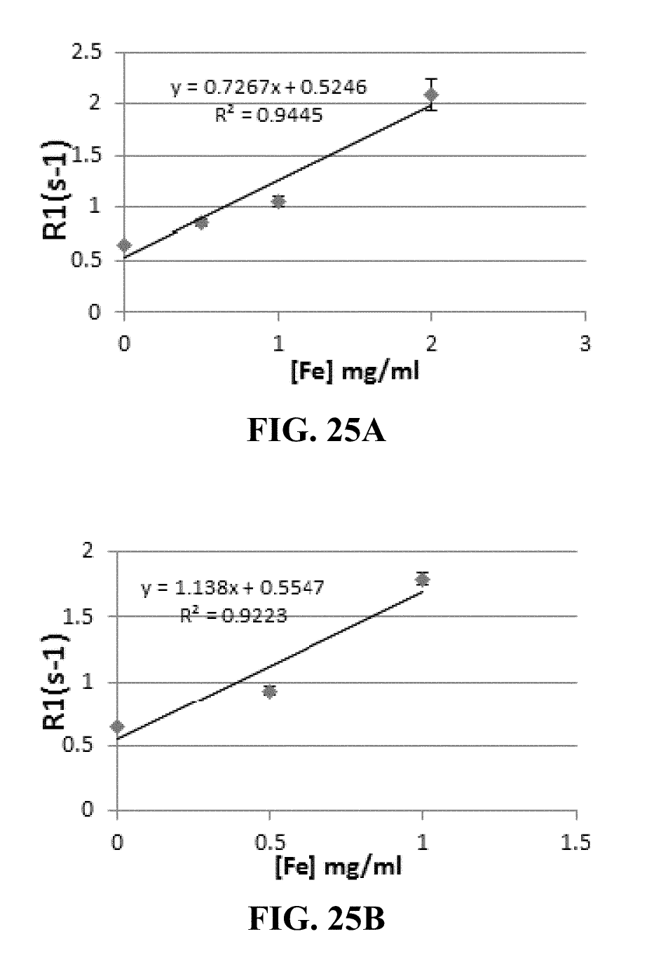

FIGS. 25A and 25B are plots of relaxivity (R1, measured in s⁻¹) versus SPION concentration for MRI visible microspheres. As shown in FIGS. 25A and 25B, the measured relaxivity increases with higher SPION concentration in the microspheres.

### Example 18: Drug Loading and Release from MRI Visible Microspheres

About 180 mg of MRI Visible Microspheres with diameters between about 300 and about 500 μm were immersed in about 0.2 mL of doxorubicin solution with a concentration of 25 mg doxorubicin per mL at room temperature. After 2 hours, the concentration of the doxorubicin was determined by absorbance measurement at 482 nm with a spectrophotometer (data not shown). The loading percentage was 99.49%.

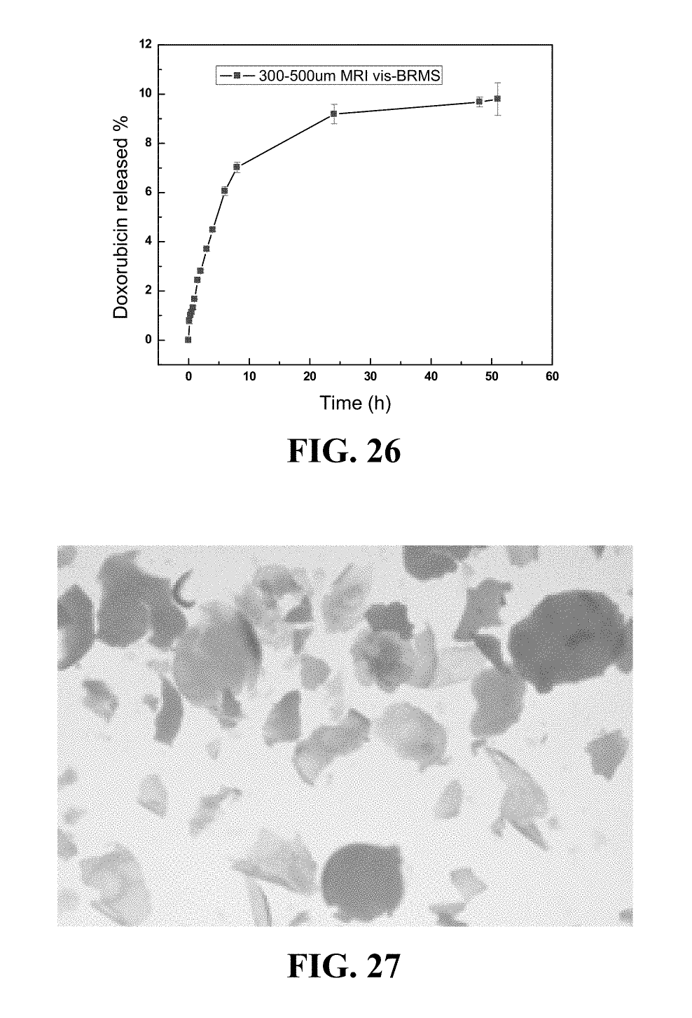

In phosphate-buffered saline (PBS), the doxorubicin-loaded MRI visible microspheres gradually release doxorubicin. MM visible microspheres containing about 0.5 mg doxorubicin were placed in a 4 mL cuvette containing about 2 mL PBS. The cuvette was placed on a shaker (50 stroke/min) at 37° C. The absorbance was measured without refreshing the medium. FIG. 26 is a plot of percent doxorubicin released versus time. As shown in FIG. 26, under this static medium, the doxorubicin release had a fast initial release period followed by a plateau. The percentage of doxorubicin released was about 9.18% at 24 hours.

### Example 19: Degradation of Doxorubicin Loaded MRI Visible Microspheres

The doxorubicin loaded MM visible microspheres were allowed to degrade in the PBS. FIG. 27 is a light microscopy image showing degraded doxorubicin loaded MM visible microspheres after 13 days in 0.01M PBS at 37° C. As shown in FIG. 27, the microspheres degraded into smaller pieces. The microspheres remained red, indicating that there was still some residual doxorubicin remained on the MRI visible microspheres since the PBS was not refreshed.

Various examples have been described. These and other examples are within the scope of the following claims.

1. An embolic material comprising:
a microsphere comprising a diameter between about 50 micrometers and about 2200 micrometers, wherein the microsphere comprises carboxymethyl chitosan crosslinked with carboxymethyl cellulose and an imaging agent integrally contained within the microsphere.

2. The embolic material of claim 1, wherein the imaging agent comprises at least one of an ethiodized oil, a radiopaque metal, or superparamagnetic iron oxide nanoparticles (SPIONs).

3. The embolic material of claim 2, wherein the imaging agent comprises about one part by weight of the ethiodized oil to about 3 parts by weight of the radiopaque metal.

4. The embolic material of claim 2, wherein the radiopaque metal is selected from the group consisting of tantalum, tungsten, barium, bismuth, gold, platinum, and combinations thereof.

5. The embolic material of claim 1, wherein the microsphere comprises about 5% to about 75% by weight of the imaging agent.

6. The embolic material of claim 2, wherein the imaging agent consists essentially of the ethiodized oil or the SPIONs.

7. The embolic material of claim 1, wherein the microsphere defines a diameter between about 50 micrometers and about 2200 micrometers.

8. The embolic material of claim 1, further comprising a therapeutic agent, wherein the therapeutic agent comprises at least one of doxorubicin, irinotecan, sunitinib, idarubicin, ambroxol, irinotecan, or epidubicin.

9. A method of forming an embolic microsphere comprising:
at least partially oxidizing carboxymethyl cellulose (CMC) to form partially oxidized CMC (OCMC);
forming an emulsion of the OCMC, carboxymethyl chitosan (CCN), an aqueous solvent, an imaging agent, and a non-aqueous solvent; and
crosslinking the CCN with the OCMC to form the embolic microsphere, wherein the imaging agent is integrally contained within the embolic microsphere.

10. The method of claim 9, wherein forming the emulsion comprises mixing the imaging agent in the aqueous solvent.

11. The method of claim 9, wherein the imaging agent comprises at least one of ethiodized oil, a radiopaque metal, or superparamagnetic iron oxide nanoparticles (SPIONs).

12. The method of claim 11, further comprising mixing the radiopaque metal with the ethiodized oil to form the imaging agent.

13. The method of claim 9, wherein crosslinking the CCN with the OCMC to form the embolic microsphere comprises mixing the emulsion at a temperature of between about 20° C. and about 70° C. for at least about 16 hours.

14. The method of claim 9, wherein crosslinking the CCN with the OCMC to form the embolic microsphere comprises crosslinking the CCN with the OCMC to form an embolic microsphere comprising a diameter between about 50 micrometers and about 2200 micrometers.

15. An embolization suspension comprising:
a solvent; and
a plurality of microspheres suspended in the solvent, wherein at least one of the plurality of microspheres comprises a diameter between about 50 micrometers and about 2200 micrometers, and wherein the at least one of the plurality of microspheres comprises carboxymethyl chitosan crosslinked with partially oxidized carboxymethyl cellulose, and an imaging agent integrally contained within the at least one microsphere.

16. The embolization suspension of claim 15, wherein the imaging agent comprises at least one of an ethiodized oil, a radiopaque metal, or superparamagnetic iron oxide nanoparticles (SPIONs).

17. The embolization suspension of claim 16, wherein the imaging agent comprises about 1 part by weight of the ethiodized oil to about 3 parts by weight of the radiopaque metal.

18. The embolization suspension of claim 15, wherein the microsphere comprises about 5% to about 75% by weight of the imaging agent.

19. The embolization suspension of claim 16, wherein the imaging agent consists essentially of the ethiodized oil or the SPIONs.

20. The embolization suspension of claim 15, further comprising a therapeutic agent, wherein the therapeutic agent comprises doxorubicin, irinotecan, sunitinib, idarubicin, ambroxol, irinotecan, or epidubicin.
